(19) United States
(12) Patent Application Publication
(10) Pub. No.: US 2023/0298722 A1
(43) Pub. Date: Sep. 21, 2023

(54) MODELS TO PREDICT MEDICATION EFFECTIVENESS

(71) Applicant: MatrixCare, Inc., Bloomington, MN (US)

(72) Inventor: Vivek KUMAR, Eden Prairie, MN (US)

(21) Appl. No.: 18/158,909

(22) Filed: Jan. 24, 2023

Related U.S. Application Data
(60) Provisional application No. 63/322,129, filed on Mar. 21, 2022.

(51) Int. Cl. G16H 20/10; G16H 10/60; G16H 50/30

(52) U.S. Cl. CPC G16H 20/10; G16H 10/60; G16H 50/30

(57) ABSTRACT

Techniques for improved machine learning are provided. Patient data describing a patient is received, and a medication to be evaluated with respect to the patient is identified. An efficacy score is generated by processing at least a subset of the patient data using a hybrid machine learning model comprising a static portion and a dynamic portion, where the efficacy score indicates predicted efficacy of the medication for the patient. The medication is provided for the patient based at least in part on the efficacy score.

### CROSS-REFERENCE TO RELATED APPLICATIONS

This application claims priority to U.S. Provisional Patent Application No. 63/322,129, filed Mar. 21, 2022, the entire content of which is incorporated herein by reference in its entirety.

### INTRODUCTION

Embodiments of the present disclosure relate to efficacy modeling. More specifically, embodiments of the present disclosure relate to using computer modeling to generate efficacy predictions.

In conventional healthcare settings, such as residential care facilities (e.g., nursing homes), hospitals, in-patient or out-patient clinics, and the like, healthcare providers review a wide variety of patient data to drive optimal treatment decisions. For example, doctors may manually review the patient's electronic health records to determine which medication(s) (which can include not only identifying a class of medication or a specific medication in the class, but also selecting the dosage and length of the prescription). Additionally, in many cases, healthcare providers are expected to understand huge amounts of information relating to the alternatives, including contraindications, side effects, medication interactions, and the like.

The vast amount of relevant information prevents effective or accurate selections, and conventional providers are generally uncertain as to whether a given medication will work for a given patient, much less how quickly it will do so. Further, even if ample data and time is available to make such decisions, conventional approaches are inherently subjective and require extensive personal experience for adequate results. This manual review frequently fails to identify the most optimal options for a variety of users. Further, given the vast complexity involved in modern healthcare, it is simply impossible for a healthcare provider to evaluate all relevant data and alternatives.

Improved systems and techniques to automatically generate and evaluate treatment efficacies.

### SUMMARY

According to one embodiment presented in this disclosure, a method is provided. The method includes: receiving patient data describing a patient; identifying a medication to be evaluated with respect to the patient; generating an efficacy score by processing at least a subset of the patient data using a hybrid machine learning model comprising a static portion and a dynamic portion, wherein the efficacy score indicates predicted efficacy of the medication for the patient; and providing the medication for the patient based at least in part on the efficacy score.

According to one embodiment presented in this disclosure, a method is provided. The method includes: receiving patient data describing a patient; identifying a medication consumed by the patient; determining an efficacy of the medication based at least in part on whether the medication was successful in treating a disorder of the patient; training a hybrid machine learning model comprising a static portion and a dynamic portion based on at least a subset of the patient data and the determined efficacy of the medication; and deploying the hybrid machine learning model.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow for training machine learning models based on historical data.

FIG. 2 depicts an example workflow for generating and evaluating medication plans using machine learning models.

FIG. 3 depicts an example workflow for refining machine learning models to predict efficacy.

FIG. 4 depicts an example workflow to generate efficacy scores using hybrid machine learning models.

FIG. 5 depicts an example workflow for preprocessing unstructured data for improved machine learning.

FIG. 6 is a flow diagram depicting an example method for training machine learning models to predict efficacy.

FIG. 7 is a flow diagram depicting an example method for predicting efficacy using machine learning models.

FIG. 8 is a flow diagram depicting an example method for extract and preprocess patient data for improved machine learning.

FIG. 9 is a flow diagram depicting an example method for preprocessing unstructured input data to improve machine learning results.

FIG. 10 is a flow diagram depicting an example method for generating efficacy scores using medication-specific machine learning models.

FIG. 11 is a flow diagram depicting an example method for generating efficacy scores using medication-agnostic machine learning models.

FIG. 12 is a flow diagram depicting an example method for refining machine learning models to generate efficacy scores.

FIG. 13 is a flow diagram depicting an example method for generating efficacy scores using trained machine learning models.

FIG. 14 is a flow diagram depicting an example method for training machine learning models to predict efficacy.

FIG. 15 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved models to evaluate patient data and predict treatment efficacies.

In some embodiments, one or more medication efficacy models (also referred to in some aspects generically as models or treatment models) can be built, generated, trained, and/or used as a tool for clinicians (e.g., nurses, caregivers, doctors, and the like) to assist in generating and evaluating medication plans for users (e.g., patients, residents of a long-term care facility, and the like), thereby improving care, and preventing potentially significant negative outcomes. In some embodiments, machine learning is used to generate efficacy scores indicating how effective a given medication course is predicted to be for a given patient. In some embodiments, the efficacy can include whether the medication will work at all, how long it will take to work, whether adverse events are expected to occur, how much time will elapse before onset of such adverse events, and the like.

In conventional settings, healthcare providers must rely on subjective assessments and plans (e.g., manually selecting medications) to care for patients in view of a variety of published literature, such as clinical studies. In addition to this inherently subjective and potentially inaccurate approach, many conventional systems are largely static and have difficultly responding to dynamic situations (e.g., changing patient conditions) or new evidence. Moreover, the vast number and variety of alternative treatments that can be used, as well as the significant amounts of data available describing each patient, render accurate analysis and selection of proper medication courses impossible to adequately perform manually or mentally. Aspects of the present disclosure can supplement or remove this subjective review and can further prevent wasted time and computational expense spent reviewing vast amounts of irrelevant data and sub-optimal courses. Further, aspects of the present disclosure enable more accurate evaluations, more efficient use of computational resources, and overall improved outcomes for patients.

Embodiments of the present disclosure can generally enable proactive and quality care for users, as well as dynamic and targeted medications, that help to prevent or reduce adverse events due to a variety of issues and conditions. This autonomous and continuous updating based on specific and (potentially) changing conditions with respect to individual patients enables a wide variety of improved results, including not only improved outcomes for the users (e.g., reduced adverse events, early identification of optimal interventions that will reduce or ameliorate the patient's conditions, and the like) but also improved computational efficiency and accuracy of the evaluation and solution process.

In some embodiments, a variety of historical patient data can be collected and evaluated to generate one or more efficacy models. This may include, for example, building rules-based or algorithmic models (e.g., static models) and/or training one or more machine learning models (including dynamically-learned machine learning models such as neural networks, and/or hybrid models having dynamic portion(s) and static portion(s)). During such creation or training, the (machine learning) model(s) can learn a set of features (e.g., attributes) and/or a set of weights for such features. These features and weights can then be used to automatically and efficiently process new user data in order to generate and evaluate medication course efficacy. In some aspects, the model may be trained during a training phase, and then be deployed as a static model that, once deployed, remains fixed. In other embodiments, the model may be refined or updated (either periodically or upon specified criteria). That is, during use in evaluating and generating in potential medication efficacy, the model may be refined based on feedback from users (such as doctors). For example, if a clinician indicates that a medication course was successful in remediating a patient's condition (regardless of whether the model suggested the medication or scored it highly), the model may be refined based on this indication. Similarly, if a clinician indicates that a medication course failed to help the condition, the model may be refined to reflect this new data.

In some embodiments, the machine learning model is a hybrid model including one or more static portions and one or more dynamic portions. Generally, the static portion(s) may include a fixed or static architecture, algorithm, formula, set of weights or other parameters, and the like, while the dynamic portion includes parameters or architectures that were learned during a training phase and/or that are refined during use. In at least one embodiment, the hybrid model uses the output of one component as input to another. For example, some or all of the input data may be passed through one or more static portions of the model, and the output of these static portion(s) can be processed as input to the dynamic portion(s) that generate an overall output for the model. Further, in some embodiments, the efficacy model is implemented as a static model (without a trained or dynamic portion), such as a rules-based model, an algorithmic model (e.g., which computes efficacy based on one or more defined algorithms with various weights or contributions for each feature), and the like. In some embodiments, the efficacy model is implemented as a trained or dynamic model (without a static portion), such as using an artificial neural network, a random forest model, and the like.

Additionally, in some embodiments, the system/model includes an adjustment component that can dynamically adjust or modify the output efficacy score based on a variety of data. In embodiments, the adjustment component may include static architectures (e.g., fixed formulas, algorithms, or rules) and/or dynamic architectures (e.g., a neural network or other machine learning architecture). In at least one embodiment, the (potentially hybrid) model can be used to generate an initial score, and the adjustment component can generate an adjustment factor (e.g., based on specific patient attributes, such as whether they've previously used this medication or a similar medication in the same class). This adjustment factor can then be used to modify or adjust the initial score in order to generate an overall predicted efficacy for the medication course.

### Example Workflow for Training Machine Learning Models Using Historical Data

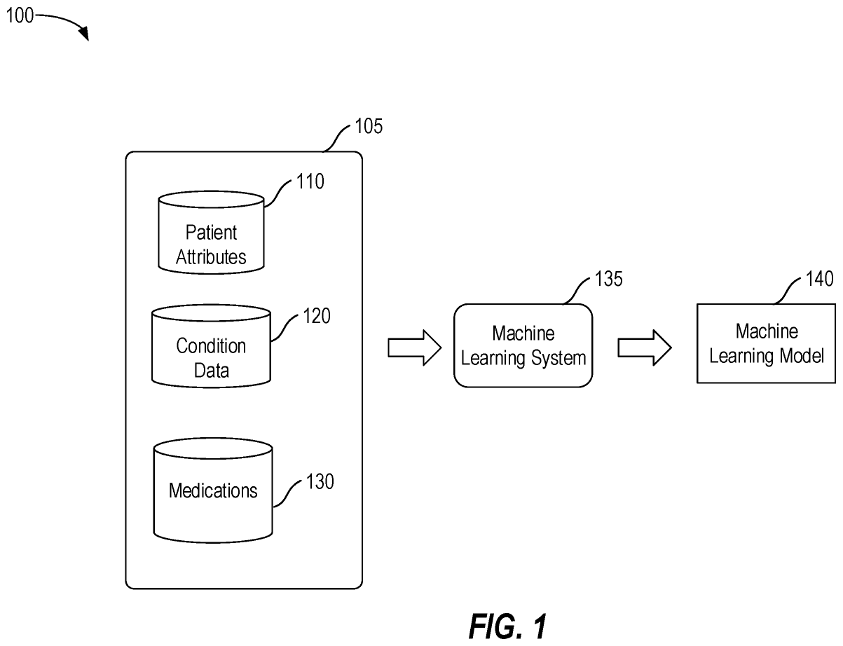

FIG. 1 depicts an example workflow 100 for training machine learning models based on historical data. Although the illustrated example depicts training a machine learning model, in some embodiments, the system can additionally or alternatively generate static model(s), and/or static portions of a hybrid machine learning model.

In the illustrated workflow 100, a set of historical data 105 (also referred to as patient data, user data, and/or resident data) is evaluated by a machine learning system 135 to generate one or more machine learning models 140 (or to generate static or rules-based models, in some embodiments). In embodiments, the machine learning system 135 may be implemented using hardware, software, or a combination of hardware and software. The historical data 105 generally includes data or information associated with one or more patients (also referred to as users or residents in some aspects) from one or more prior points in time. That is, the historical data 105 may include, for one or more patients, a set of one or more snapshots of the resident's characteristics or attributes at one or more points in time. In some embodiments, the historical data 105 includes information for patients of one or more healthcare facilities or professionals, residents residing in one or more long-term care facilities, and the like. The historical data 105 may generally be stored in any suitable location. For example, the historical data 105 may be stored within the machine learning system 135, or may be stored in one or more remote repositories, such as in a cloud storage system.

In the illustrated example, the historical data 105 includes, for each individual reflected in the data, a set of one or more patient attributes 110, condition data 120, and medications 130. In some embodiments, as discussed above, the historical data 105 includes data at multiple points in time for each patient. That is, for a given patient, the historical data 105 may include multiple sets of patient attributes 110 (one set for each relevant point in time), and the like. In some embodiments, the data contained within the patient attributes 110, condition data 120, and medications 130 are associated with timestamps or other indications of the relevant time or period for the data. In this way, the machine learning system 135 can identify the relevant data for any given point or window of time. For example, for a given course of medications 130 at a given time, the machine learning system 135 can identify all the relevant data surrounding this time (e.g., the patient attributes 110 and/or condition data 120 at the time the plan was instantiated, within a predefined window before the time, such as one month prior, and the like).

In some embodiments, the historical data 105 may be collectively stored in a single data structure. For example, the patient attributes 110, condition data 120, and/or medications 130 may each be represented in electronic health records (EHR) and/or in a patient profile, or as a sequence of structures (e.g., a set of profiles or forms, each corresponding to a particular point or window in time and containing attributes for that time). In some portions of the present discussion, the various components of the historical data 105 are described with reference to a single patient for conceptual clarity (e.g., patient attributes 110 of a single patient). However, it is to be understood that the historical data 105 can generally include such data for any number of patients.

As discussed in more detail below, the patient attributes 110 generally correspond to a set of one or more specified features, attributes, or characteristics describing the patient(s). For example, the patient attributes 110 may include characteristics such as patient age, the biological or assigned sex of the patient, allergies the patient has, diagnoses or disorders they have, medications the patient uses, assistance they require (e.g., whether they need assistance walking), and the like. In at least one embodiment, the patient attributes 110 can include information or data generated by machine learning models or other techniques. For example, the patient attributes 110 may include information that was extracted from, or generated based on, natural language text (e.g., progress notes) describing the patient, as discussed in more detail below.

In some embodiments, the historical data 105 can indicate, for each specified feature, whether the corresponding resident has the attribute (or the value of the attribute) at the relevant time. In some embodiments, the patient attributes 110 are curated or selected based on their impact on how patients respond to various medical interventions. For example, in one aspect, a user (e.g., a clinician) may manually specify attributes that have a high impact on the success of various medication courses. In some embodiments, some or all of the attributes may be inferred or learned (e.g., using one or more feature selection techniques). For example, one or more machine learning models or feature selection algorithms may be used to identify specific attributes (or to determine dynamic weights for each attribute) based on their impact on the success of medication plans.

As discussed in more detail below, the condition data 120 generally corresponds to information relating to a set of specified diagnoses, diseases, assessments, problems, conditions, disorders, or issues relating to the functional state of the resident. For example, the condition data 120 may indicate disorders experienced by or reported by the user or a clinician, such as whether the patient has the given disorder at the given time. The condition data 120 can generally indicate, for each condition, whether the corresponding resident has the specified condition at the time, previously had the condition, and the like. In some embodiments, the condition data 120 can further indicate details about each condition, such as the severity, time of onset, time of remediation (if it was remediated), and the like. In some embodiments, the condition data 120 includes data for each problem or issue that the machine learning system 135 is configured to evaluate. That is, for each possible condition for which a medication course can or should be generated by the machine learning system 135, the condition data 120 may include data for the condition.

As discussed in more detail below, the medications 130 can generally indicate a set of details for how clinicians, caregivers, nurses, doctors, or other users previously treated or otherwise responded to each condition of the patient using a course of medication(s). In one such aspect, for each medication that the patient has been prescribed, the medications 130 may indicate information such as the identity of the medication, the class of the medication, what condition(s) the medication was intended to resolve, the dosage and/or timing of the course (e.g., how many doses per day, the size of each dose, the number of days in the course, and the like), and the like. In some embodiments, the medications 130 (or other data, such as the condition data 120 or patient attributes 110) further indicate whether any medications have been discontinued for the patient. Further, in some embodiments, the information indicates, for each discontinued medication, why the medication was discontinued. In at least one embodiment, the historical data 105 indicates at least whether the medication was discontinued due to adverse reaction or due to the condition being resolved. In some embodiments, the data may include other reasons such as that the medication was a data entry error, that the patient was discharged, that the medication was not working, and the like.

In an embodiment, the medications 130 are selected by clinicians (e.g., nurses or doctors) for the resident. That is, the medications 130 represent historical courses of treatment that were defined by users to treat or mitigate prior conditions for one or more patient. As discussed below in more detail, the machine learning system 135 can generally evaluate these historical medications 130 and other data in order to determine how effective they were (e.g., whether they remediated the patient condition(s), how quickly the issue resolved, and the like). One or more machine learning models 140 can then be trained to generate and/or score new medication treatment plans to indicate whether they should be used (e.g., how likely they are to remediate a condition given the specific attributes of the patient, how long it will likely take, and the like) based on various patient attributes.

Although the illustrated historical data 105 includes several specific and discrete components including patient attributes 110, condition data 120, and medications 130, in some embodiments, the historical data 105 used by the machine learning system 135 may include fewer components (e.g., a subset of the illustrated examples) or additional components not depicted. Additionally, though the illustrated example provides general groupings of data to aid understanding, in some embodiments, the historical data 105 may be represented using any number of groups. For example, the condition data 120 and/or medications 130 may be reflected in EHR included in the patient attributes 110.

As illustrated, the machine learning system 135 generates one or more machine learning models 140 based on the historical data 105. The machine learning model 140 generally specifies a set of weights for the various features or attributes of the historical data 105. In some embodiments, the machine learning model 140 specifies weights specifically for each individual feature (e.g., for each attribute in the set of patient attributes 110). For example, a first attribute may be associated with a lower weight than a second attribute. Similarly, in some embodiments, the machine learning model 140 specifies different weights depending on the severity of the feature (e.g., depending on the severity of a disorder or diagnosis indicated in the condition data 120).

Though the illustrated example is labeled a “machine learning” system generating a “machine learning model”, in some embodiments, the system may additionally or alternatively generate a static efficacy model. For example, as discussed above, the static model may comprise one or more rules or algorithms used to compute an efficacy score for medications. As one example, one or more rule(s) may indicate when particular medications are appropriate or inappropriate, and one or more algorithms (specifying various weights for various patient features) may be used to compute predicted efficacy for particular medications based on those patient features.

In some embodiments, the specific features considered by the machine learning model 140 (e.g., the specific patient attributes 110) are manually defined and curated. For example, the specific features may be defined by a subject-matter expert. In other embodiments, the specific features are learned during a training phase.

For example, the machine learning system 135 may process the historical data 105 for a given patient at a given time as input to the machine learning model 140 in order to generate a predicted efficacy of a given medication to treat a diagnosis of the patient. This predicted efficacy can then be compared against ground-truth data (e.g., determined based on whether the patient actually recovered, as indicated in the historical data 105). The difference between the predicted and actual recovery can be used to refine the weights of the machine learning model 140, and the model can be iteratively refined (e.g., using data from multiple patients and/or multiple points in time) to accurately evaluate medication courses or plans.

In some embodiments, during or after training, the machine learning system 135 may prune the machine learning model 140 based in part on the learned weights. For example, if the learned weight for a given feature (e.g., a specific patient attribute 110) is below some threshold (e.g., within a threshold distance from zero), the machine learning system 135 may determine that the feature has no impact (or negligible impact) on the efficacy of care plans. Based on this determination, the machine learning system 135 may cull or remove this feature from the machine learning model 140 (e.g., by removing one or more neurons, in the case of a neural network). For future evaluations, the machine learning system 135 need not receive data relating to these removed features (and may refrain from processing or evaluating the data if it is received). In this way, the machine learning model 140 can be used more efficiently (e.g., with reduced computational expense and latency) to yield accurate evaluations.

In some embodiments, the machine learning system 135 can generate multiple machine learning models 140. For example, a separate machine learning model 140 may be generated for each condition or diagnosis (e.g., with a unique model for each specific diagnosis), for each medication (e.g., with a unique model trained to predict efficacy of a specific medication or class of medications), and the like. This may allow the machine learning system 135 to account for a variety of disorder-specific, medication-specific, or other specific changes or peculiarities. In other embodiments, the machine learning system 135 generates a universal machine learning model 140. In at least one embodiment, the machine learning model 140 may use similar considerations (e.g., location, region, and the like) as an input feature.

In some embodiments, the machine learning model 140 is a hybrid model having one or more static portions and one or more dynamic portions. Generally, the static portion may correspond to a set of weights, algorithms, rules, or other fixed architectures configured to process some or all of the input data. The dynamic portion may correspond to dynamic or changing weights, architectures, algorithms, or other architectures. For example, the dynamic portion(s) of the machine learning model 140 may use trained weights (learned during a training phase) that may vary or adjust the impact of input features during inferencing, while the static portion(s) include fixed weights.

In at least one embodiment, the output of the static portion(s) of the model are used as input to the dynamic portion(s). For example, some of the patient attributes 110 such as age and sex may be provided directly as input to the dynamic portion (e.g., to a neural network), while others (such as natural language notes) may be first processed using the static portion (e.g., to perform keyword extraction, normalization, and the like). The output of this static portion of the machine learning model 140 can then be provided as input to the dynamic portion (e.g., as input to a neural network). This hybrid approach can result in improved computational efficiency (e.g., reducing the expense of using the model), improved model accuracy (resulting in more accurate efficacy predictions), and the like.

Additionally, in some embodiments, the machine learning model 140 may include an adjustment portion or component that is configured to modify the generated efficacies based on a subset of the input data. For example, the machine learning model (which may include both a static portion and a dynamic portion) may generate an initial efficacy score indicating the predicted efficacy of a given medication in treating a given disorder of the specific patient. The adjustment portion may then evaluate a defined subset of data (such as medications that the patient has previously used) to generate an adjustment factor which is used to modify the initial score (e.g., by scaling it up or down).

For example, if the medication data 130 indicates that the patient already used the specified medication (or another medication in the same class) previously, the adjustment component may generate an adjustment factor that reduces the initial efficacy score (e.g., to indicate that the specific medication course is less likely to work).

In some embodiments, the machine learning system 135 outputs the machine learning model 140 to one or more other systems for use. That is, the machine learning system 135 may distribute the machine learning model 140 to one or more downstream systems, where each downstream system can use the model to predict medication efficacies. For example, the machine learning system 135 may deploy the machine learning model 140 to one or more servers associated with specific care facilities or hospitals, and these servers may use the model to evaluate medication courses for patient of the specific facility. In at least one embodiment, the machine learning system 135 can itself use the machine learning model to evaluate medication efficacy across one or more locations.

### Example Workflow for Evaluating Medication Plans Using Machine Learning

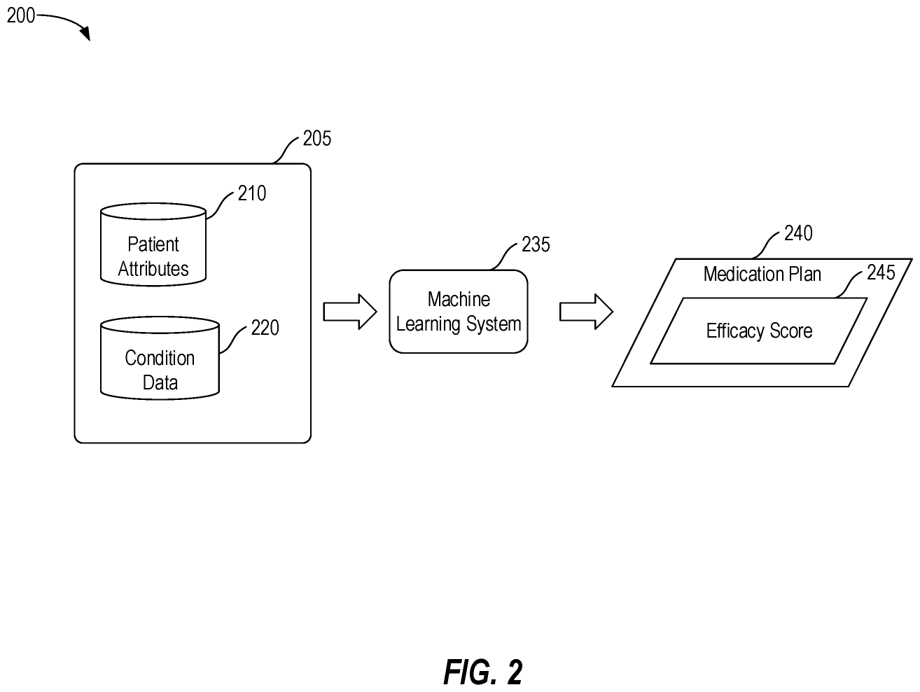

FIG. 2 depicts an example workflow 200 for generating and evaluating medication plans using machine learning models. In some embodiments, the workflow 200 is performed using trained models, such as the machine learning models 140 of FIG. 1.

In the illustrated workflow 200, a set of patient data 205 is evaluated by a machine learning system 235 using one or more machine learning models (e.g., machine learning model 140 of FIG. 1) to generate and/or evaluate one or more medication plan(s) 240, each with a corresponding efficacy score 245. In embodiments, the machine learning system 235 may be implemented using hardware, software, or a combination of hardware and software. In some embodiments, the machine learning system 235 corresponds to the machine learning system 135 of FIG. 1. In other aspects, as discussed above, the machine learning system 235 may differ from the system that trained the model and/or generated the training data. Further, as discussed above, though depicted and described as a machine learning system, in some embodiments, the system may additionally or alternatively use purely static-models (e.g., rules-based and/or algorithmic models), or may use a hybrid combination of static and dynamic models.

The patient data 205 generally includes data or information associated with one or more patient (also referred to as residents or users). That is, the patient data 205 may include, for one or more patient, a set of one or more snapshots of the patient characteristics or attributes at one or more points in time. The patient data 205 may generally be stored in any suitable location. For example, the patient data 205 may be stored within the machine learning system 235, or may be stored in one or more remote repositories, such as in a cloud storage system. In at least one embodiment, the patient data 205 is distributed across multiple data stores.

In the illustrated example, the patient data 205 includes, for each patient reflected in the data, a set of one or more patient attributes 210 and condition data 220. In some embodiments, as discussed above, the data contained within the patient attributes 210 and patient data 220 are associated with timestamps or other indications of the relevant time or period for the data. In this way, the machine learning system 235 can generate a corresponding medication plan 240 based on the relevant attributes 210 and conditions 220 at a given time.

As discussed above, the patient attributes 210 generally include information relating to a set of one or more specified features, attributes, or characteristics describing the patient(s) (such as demographic data, allergy data, diagnoses of the patient, other medications used by the patient, and the like), and the condition data 220 generally corresponds to information relating to a set of specified diagnoses, assessments, problems, conditions, disorders, or issues of the patient.

Although the illustrated patient data 205 includes several discrete components for conceptual clarity, in some embodiments, the patient data 205 used by the machine learning system 235 may include fewer components (e.g., a subset of the illustrated examples) or additional components not depicted. Additionally, though the illustrated example provides general groupings of attributes to aid understanding (e.g., grouping attributes and conditions), in some embodiments, the patient data 205 may be represented using any number of groups.

Additionally, though the above discussion relates to receiving specified sets of data (e.g., specified diagnoses or other attributes), in some aspects, the machine learning system 235 may receive a broader set of data (e.g., all diagnoses or attributes of the patients) and select which subset of the data to consider (e.g., based on the features specified in the machine learning model).

In the illustrated example, the patient data 205 is used to generate one or more medication plans 240, each with a corresponding efficacy score 245. As discussed above, the medication plans 240 can generally indicate a set of details for a course of one or more medications to be used to treat the patient. For example, for a diagnosis reflected in the condition data 220, the medication plan 240 may indicate a class of medication and/or a specific medication, a dosage, a timing, a length or duration of the course, and the like.

In at least one embodiment, the machine learning system 235 generates the medication plans 240 and efficacy scores 245 by processing a variety of alternatives (sequentially or in parallel). For example, for each of a defined set of potential medication treatments (e.g., defined in the literature) for a given disorder, the machine learning system 235 may generate a corresponding score indicating the suitability of the medication (or, in some embodiments, for each specific course of dosage, time, duration, and the like). In some embodiments, a user (e.g., a doctor) may specify the proposed medication plan 240, and the machine learning system 235 can generate a corresponding efficacy score 245 indicating the likely efficacy of the plan. These score(s) can then be used to define the efficacy score 245 for each alternative medication plan 240.

As discussed above, the machine learning system 235 can generate the efficacy scores 245 by identifying or extracting the relevant attributes from the patient data 205 (e.g., the relevant demographics of the patient, as indicated by the efficacy model), receiving an indication of the proposed medication plan, and processing these attributes using the weights and architecture of the (dynamic, static, or hybrid) model to generate an overall efficacy score 245 for the user based on the attributes and plan. In some embodiments, the efficacy scores 245 can additionally or alternatively include a classification or category, such as a low, moderate, or high probability of success, or a short-term, medium-term, or long-term expected duration needed, determined based on one or more threshold values for the recovery scores.

In some embodiments, the machine learning system 235 uses a diagnosis-agnostic model that can be used to generate efficacy scores for a variety of disorders. In some embodiments, the machine learning system 235 uses a set of diagnosis-specific models, where each model is used to generate efficacy scores for a specific disorder. In some embodiments, the machine learning system 235 uses a medication-agnostic model that can be used to generate efficacy scores for a variety of medications or medication classes. In some embodiments, the machine learning system 235 uses a set of medication-specific models, where each model is used to generate efficacy scores for a specific medication or set of medications.

In at least one embodiment, the alternative plans can be ranked and/or filtered based on the efficacy scores 245, and one or more top-scored medication plans 240 can be selected. These top plans may be suggested or recommended to a user (e.g., output to a clinician via a graphical user interface (GUI)), automatically initiated, and the like.

In some embodiments, the machine learning system 235 can generate a new efficacy score 245 and/or medication plan 240 for each patient periodically (e.g., daily). In some embodiments, the machine learning system 235 generates a new medication plan 240 and efficacy score 245 whenever new data becomes available (or when the patient data 205 changes). For example, when the patient attribute(s) change, the machine learning system 235 may use the updated attributes to generate a new medication plan 240. In some embodiments, whenever a patient's attributes change (e.g., due to a newly-received diagnosis), the machine learning system 235 may automatically detect the change and generate an updated medication plan 240 that is specifically-tailored to the individual resident at the specific time. This targeted prophylactic treatment can significantly improve patient conditions.

Advantageously, the automatically generated medication plans 240 and efficacy scores 245 can significantly improve the outcomes of the patients, helping to identify optimal treatment options, thereby preventing further deterioration and significantly reducing harm. Additionally, the autonomous nature of the machine learning system 235 enables improved computational efficiency and accuracy, as the efficacy scores 245 and/or medication plans 240 are generated objectively (as opposed to the subjective judgment of clinicians or other users), as well as quickly and with minimal computational expense. That is, as the scores and plans can be automatically updated whenever new data is available, users need not manually retrieve and review the relevant data (which incurs wasted computational expense, as well as wasted time for the user).

Further, in some embodiments, the machine learning system 235 can regenerate medication plans 240 and/or efficacy scores 245 during specified times (e.g., off-peak hours, such as overnight) to provide improved load balancing on the underlying computational systems. For example, rather than requiring caregivers to retrieve and review patient data repeatedly to define new medication plans, the machine learning system 235 can automatically identify such changes, and use the machine learning model(s) to regenerate medication plans 240 and efficacy scores 245. This can transfer the computational burden, which may include both processing power of the storage repositories and access terminals, as well as bandwidth over one or more networks, to off-peak times, thereby reducing congestion on the system during ordinary (e.g., daytime) use and taking advantage of extra resources that are available during the non-peak (e.g., overnight) hours.

In these ways, embodiments of the present disclosure can significantly improve patient outcomes while simultaneously improving the operations of the computers and/or networks themselves (at least through improved and more accurate scores and plans, as well as better load balancing of the computational burdens). Additionally, in some embodiments, use of a hybrid model can further improve the computational efficiency and accuracy, resulting in reduced latency, reduced computational resource consumption, and more effective medication plans.

### Example Workflow for Refining Machine Learning Models

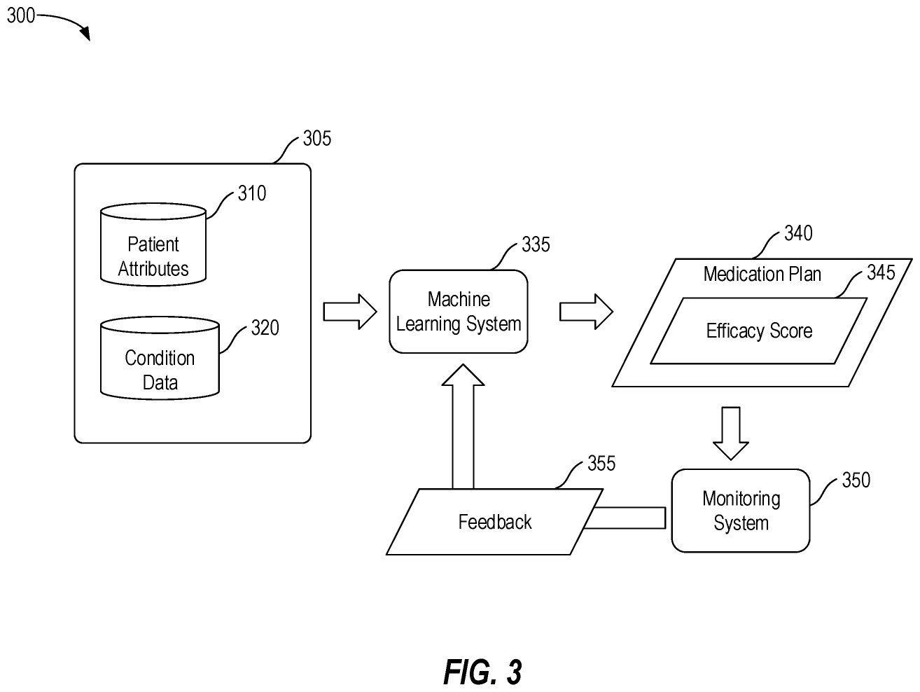

FIG. 3 depicts an example workflow 300 for refining machine learning models to predict efficacy. In some embodiments, the workflow 300 is performed using trained models, such as the machine learning models 140 of FIG. 1.

The depicted workflow 300 differs from the workflow 200 of FIG. 2 in that it enables online refinement of the machine learning models based on updated information, thereby ensuring that the models can dynamically create optimized plans even when general conditions change. In the illustrated workflow 300, a set of patient data 305 is evaluated by a machine learning system 335 using one or more efficacy models (e.g., machine learning model 140 of FIG. 1, a hybrid model, a static model, and the like) to generate and/or evaluate one or more medication plan(s) 340, each with a corresponding efficacy score 345. In embodiments, the machine learning system 335 may be implemented using hardware, software, or a combination of hardware and software. In some embodiments, the machine learning system 335 corresponds to the machine learning system 135 of FIG. 1, and/or the machine learning system 235 of FIG. 2. In other aspects, as discussed above, the machine learning system 335 may differ from the system that trained the model and/or generated the training data.

In some embodiments, the workflow 300 generally mirrors the workflow 200 of FIG. 2 to generate the medication plans 340 and efficacy scores 345. As discussed above, the patient data 305 generally includes data or information associated with one or more patients (also referred to as residents or users). That is, the patient data 305 may include, for one or more patients, a set of one or more snapshots of the patient characteristics or attributes at one or more points in time. The patient data 305 may generally be stored in any suitable location. For example, the patient data 305 may be stored within the machine learning system 335, or may be stored in one or more remote repositories, such as in a cloud storage system. In at least one embodiment, the patient data 305 is distributed across multiple data stores.

In the illustrated example, the patient data 305 includes, for each patient reflected in the data, a set of one or more patient attributes 310 and condition data 320. In some embodiments, as discussed above, the data contained within the patient attributes 310 and condition data 320 are associated with timestamps or other indications of the relevant time or period for the data. In this way, the machine learning system 335 can generate a corresponding medication plan 340 based on the relevant attributes 310 and conditions 320 at a given time.

As discussed above, the patient attributes 310 generally include information relating to a set of one or more specified features, attributes, or characteristics describing the patient(s) (such as demographic data, allergy data, diagnoses of the patient, other medications used by the patient, and the like), and the condition data 320 generally corresponds to information relating to a set of specified diagnoses, assessments, problems, conditions, disorders, or issues of the a.

Although the illustrated patient data 305 includes several discrete components for conceptual clarity, in some embodiments, the patient data 305 used by the machine learning system 335 may include fewer components (e.g., a subset of the illustrated examples) or additional components not depicted. Additionally, though the illustrated example provides general groupings of attributes to aid understanding (e.g., grouping attributes and conditions), in some embodiments, the patient data 305 may be represented using any number of groups (e.g., the condition data 320 may be reflected within the patient attributes 310).

Additionally, though the above discussion relates to receiving specified sets of data (e.g., specified diagnoses or other attributes), in some aspects, the machine learning system 335 may receive a broader set of data (e.g., all diagnoses or attributes of the patient) and select which subset of the data to consider (e.g., based on the features specified in the machine learning model).

In the illustrated example, the resident data 305 is used to generate one or more medication plans 340, each with a corresponding efficacy score 345. As discussed above, the medication plan 340 can generally indicate a treatment or therapy course for the patient, such as which medication(s) or class(es) of medication to use, the timing, duration, and/or dosage to use, and the like.

In at least one embodiment, the machine learning system 335 generates the medication plans 340 and efficacy scores 345 for each disorder or diagnosis by processing a variety of alternatives (sequentially or in parallel), as discussed above. In some embodiments, the machine learning system 335 can process a specific medication plan (or set of plans) provided by the user (e.g., a doctor) to generate corresponding efficacy scores 345. In at least one embodiment, the user may indicate one or more aspects of the plan (e.g., which class(es) of medications they wish to try, or what duration they want to target), and allow the machine learning system 335 to evaluate various alternative plans fitting the criteria (e.g., different drugs within the class(es), different dosages and durations, and the like).

As discussed above, the machine learning system 335 can generate the efficacy scores 345 by identifying or extracting the relevant attributes from the patient data 305 (e.g., the relevant demographics, as indicated by the model), receiving an indication of the proposed medication plan, and processing these attributes using the weights and architecture of the machine learning model to generate an overall efficacy score 345 for the user based on the attributes and plan.

In some embodiments, the machine learning system 335 uses a diagnosis-agnostic model that can be used to generate efficacy scores for a variety of disorders. In some embodiments, the machine learning system 335 uses a set of diagnosis-specific models, where each model is used to generate efficacy scores for a specific disorder. In some embodiments, the machine learning system 335 uses a medication-agnostic model that can be used to generate efficacy scores for a variety of medications or medication classes. In some embodiments, the machine learning system 335 uses a set of medication-specific models, where each model is used to generate efficacy scores for a specific medication or set of medications.

In some embodiments, the machine learning system 335 can generate a new efficacy score 345 and/or medication plan 340 for each patient periodically (e.g., daily), whenever new data becomes available, or when the patient data 305 changes.

Advantageously, the automatically generated medication plans 340 and efficacy scores 345 can significantly improve the outcomes of the patients, as discussed above, helping to identify optimal treatment options, thereby preventing further deterioration and significantly reducing harm. Additionally, the autonomous nature of the machine learning system 335 enables improved computational efficiency and accuracy, as the efficacy scores 345 and/or medication plans 340 are generated objectively (as opposed to the subjective judgment of clinicians or other users), as well as quickly and with minimal computational expense. That is, as the scores and plans can be automatically updated whenever new data is available, users need not manually retrieve and review the relevant data (which incurs wasted computational expense, as well as wasted time for the user).

Further, in some embodiments, the machine learning system 335 can regenerate medication plans 340 and/or efficacy scores 345 during specified times (e.g., off-peak hours, such as overnight) to provide improved load balancing on the underlying computational systems. For example, rather than requiring caregivers to retrieve and review patient data repeatedly to define new medication plans, the machine learning system 335 can automatically identify such changes, and use the machine learning model(s) to regenerate medication plans 340 and efficacy scores 345. This can transfer the computational burden, which may include both processing power of the storage repositories and access terminals, as well as bandwidth over one or more networks, to off-peak times, thereby reducing congestion on the system during ordinary (e.g., daytime) use and taking advantage of extra resources that are available during the non-peak (e.g., overnight) hours.

Additionally, in some embodiments, use of a hybrid model can further improve the computational efficiency and accuracy, resulting in reduced latency, reduced computational resource consumption, and more effective medication plans.

In the illustrated workflow 300, once a medication plan 340 has been implemented (e.g., once it has been selected or approved by a clinician), a monitoring system 350 can evaluate its efficacy in real-time (or near real-time). For example, the monitoring system 350 may periodically or continuously evaluate the patient data 305 (e.g., the condition data 320) to determine whether any condition(s) have changed. In at least one embodiment, while performing the medication plan (e.g., providing the indicated medications), clinicians or caregivers can note the patient's progress, such as via natural language notes, or by selecting options (e.g., from a drop down or radio button in a GUI) indicating the status of the patient and/or conditions (e.g., whether the medication plan has been discontinued). Although depicted as a discrete system for conceptual clarity, in embodiments, the monitoring system 350 may be implemented as a standalone service, or as part of the machine learning system 335 (or another system).

In the illustrated workflow, the monitoring system 350 generates feedback 355 based on the monitored medication plan(s) 340. In one embodiment, the monitoring system 350 can receive updates from users (e.g., clinicians) indicating whether the medication plan is working. For example, based on progress notes or other indications as to whether the medication plan has been discontinued, the monitoring system 350 can determine whether it succeeded or failed, whether it is ongoing, whether the patient condition is improving or worsening, and the like.

In some embodiments, the monitoring system 350 generates new feedback 355 each time the condition of a patient changes. For example, if the patient data 305 indicates that a given condition has improved or has been remediated (e.g., a diagnosis is cured), the monitoring system 350 may generate feedback 355 indicating the corresponding medication plan being used for the patient, how long the change took to occur, and the like. Similarly, if the monitoring system 350 determines that the condition has worsened or the medication plan has been discontinued, the feedback 355 can similarly indicate the corresponding medication plan, any reported adverse events, and the like.

In at least one embodiment, the monitoring system 350 may also generate feedback 355 indicating that a patient condition has not changed. For example, if the patient data 305 indicates that a given condition has remained the same for a threshold period of time (e.g., no change for a week, a month, and the like), the monitoring system 350 can generate feedback 355 indicating that the current medication plan, though not harming the patient, does not appear to be helping them recover. In some embodiments, the threshold time used may be specified based on the underlying condition, and/or may be determined based on the timeline indicated in the medication plan. For example, if the medication plan indicates a duration of one month, the monitoring system 350 may determine that the medication plan has failed once one month has passed without change.

In the illustrated workflow 300, the machine learning system 335 uses the feedback 355 to refine the efficacy model(s) used to generate and/or score medication plans. For example, if the feedback 355 indicates that a specific patient has recovered from a given condition, the machine learning system 335 may use the patient's attributes, along with the current medication plan being used for the condition, as input to the model in order to generate a new efficacy score 345 for the plan. The machine learning system 335 can then compare this generated score to the ground-truth (e.g., based on the newly-learned fact that the medication plan worked), and use the resulting difference to refine the machine learning model(s). In this way, the machine learning system 335 can learn to generate better and more accurate medication plans 340 and efficacy scores 345 for future conditions. In some embodiments, static efficacy models (or static portions of hybrid models) may similarly be updated based on the automatically-generated feedback 355 (e.g., by a user adjusting the weights and rules).

In some embodiments, as discussed above, the machine learning system 335 may perform this model refinement whenever new feedback 355 is received. In other embodiments, the machine learning system 335 may defer the refinement until specified hours (e.g., overnight, or during non-peak times) to reduce the computational burden of the refinement. In one such embodiment, the machine learning system 335 (or another system) can store the feedback 355 to be periodically used during such defined times.

### Example Workflow for Predicting Efficacy Using Hybrid Machine Learning Models

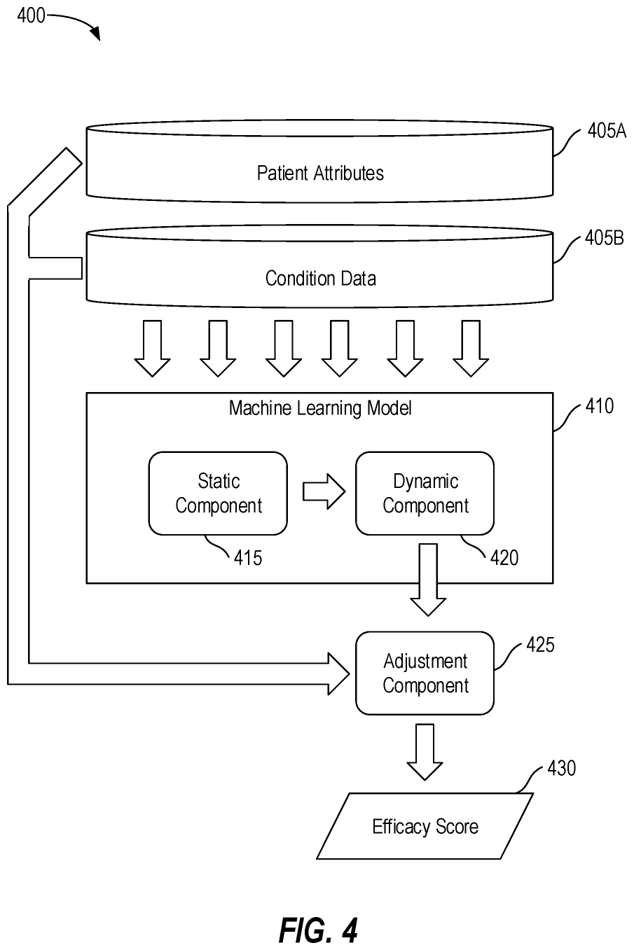

FIG. 4 depicts an example workflow 400 to generate efficacy scores using hybrid machine learning models. In some embodiments, the workflow 400 is performed by a machine learning system, such as the machine learning system 135 of FIG. 1, the machine learning system 235 of FIG. 2, and/or the machine learning system 335 of FIG. 3.

The illustrated workflow 400 provides additional detail for use of a hybrid machine learning model 410 to generate efficacy scores 430. As illustrated, a set of patient attributes 405A and condition data 405B are received as input for the machine learning model 410. The patient attributes 405A can generally include a variety of attributes or characteristics of the patient, such as demographics data (e.g., their age and/or sex), what allergies the patient has (if any), a list of medication(s) used by the patient (if any), a list of assistance actions (e.g., activities of daily living (ADL)) needed by the patient (if any) such as assistance with walking, bathing, toileting, transferring, dressing, and the like.

The condition data 405B may generally indicate the condition(s), such as diagnoses (if any) of the patient. Though depicted as discrete components for conceptual clarity, in some embodiments, the condition data 405B and patient attributes 405A may be combined and/or represented collectively (e.g., in EHR data for the patient).

As illustrated, the patient attributes 405A and condition data 405B are provided to a machine learning model 410 for processing. In some aspects, only a subset of the patient attributes 405A and condition data 405B are provided for processing by the machine learning model 410. For example, the machine learning system may select or extract a subset of attributes or conditions for processing (e.g., based on feature selection techniques, and/or based on what features the machine learning model 410 specifically uses).

In the illustrated example, the machine learning model 410 is a hybrid model having at least one static component 415 and at least one dynamic component 420. As discussed above, the static component 415 may generally correspond to fixed or static processing, such as using defined algorithms, fixed weights, and the like. The dynamic component 420 may generally correspond to dynamic processing, such as using a trained neural network having learned weights. Though illustrated as residing within the machine learning model 410, in some embodiments, the machine learning model 410 may correspond to solely the dynamic component(s) 420, and the static component(s) 415 may be used as external components (e.g., that provide input to the model, or process output from the model).

As illustrated, the static component 415 may produce output that is used as input by the dynamic component 420. That is, the static component 415 may receive some subset of the input data (e.g., specific patient attributes 405A or condition data 405B), generate an output using one or more static techniques, and provide this output as input to the dynamic component 420. The dynamic component 420 may receive a subset of the patient attributes 405A and condition data 405B, as well as the output of the static component 415, as input.

In some embodiments, the specific data used by the static component 415 may be defined (e.g., by a user) based on their impact on medication efficacy. That is, specific attributes having a particularly high impact (e.g., above a threshold) may be processed first by the static component 415. For example, the static component 415 may be configured to process significant attributes such as the current severity of the condition or specific demographic data such as age, as these may have a high impact on the efficacy of the medication plan. The dynamic component 420 may process other data where the impact is less clear (e.g., the patient's sex, other medications or diagnoses, and the like), which may be more amenable to such dynamic processing.

In at least one embodiment, the static component 415 (or another component) can include preprocessing for some or all of the data. For example, if the patient attributes 405A include natural language notes (e.g., progress notes authored by a doctor), the static component 415 may be configured to perform preprocessing such as text extraction, normalization, lemmatization, and the like, as discussed in more detail below. As a further example, the static component 415 may perform processing such as keyword identification and extraction, counting the number of instances of one or more keywords, and the like. This information (e.g., the number of times each keyword appeared in the data) may be provided as output from the static component 415. Further, though depicted as a hybrid architecture, in some embodiments, the efficacy model can be implemented using only the static component 415. That is, the model may itself comprise only static (e.g., rules-based or algorithmic) models, without the dynamic component 420 (and with or without the adjustment component 425).

In some embodiments, preprocessing the data can include encoding it for input to the model. That is, as many machine learning model architectures cannot directly work with categorical variables, the system may encode them into a numeric from. For some attributes where there is a limited number of possible unique values (such as sex, race, frequency data, and the like), the system may use one-hot encoding with a separate column for each value. However, this is not the case for attributes where there may be many (e.g., thousands) of unique values, such as a list of diagnoses, and patient allergies, and the like. One-hot encoding such data would exponentially increase the dimensionality of the data, and the machine learning model may fail due to such dimensionality.

In some embodiments, the machine learning system uses a variety of encoding techniques that are specialized for high cardinality attributes to achieve better and more efficient model performance. For example, weight of evidence (WOE) encoding and/or supervised ratio (SR) encoding may be used. These methods can take the ratio of positive and negative occurrences into account for each class in order to assign a score to each record, allowing for more efficient processing.

Though not depicted in the illustrated example, in some embodiments, the machine learning model 410 may additionally receive medication plan data as input. For example, alongside the patient attributes 405A, the machine learning model 410 may process data such as the class of medication being considered, the specific medication being considered, the timing, duration, and/or dosage being considered, and the like.

In at least one embodiment, as discussed above, the machine learning model 410 may be disorder-agnostic, disorder-specific, medication-agnostic, and/or medication-specific. For example, a medication-specific and disorder-specific model may be configured to receive patient attributes (and, in some cases, details of the specific medication course, such as dosage and duration), without receiving the specific disorder or medication as input. As the model is specific to the disorder and medication, the generated output can indicate the probability that the corresponding medication will be successful in treating the disorder (based on the patient attributes and/or dosage data). Similarly, a medication-agnostic model may receive an indication of the proposed medication (to predict efficacy of the specific medication), while a disorder-agnostic model may receive an indication as to the condition or disorder being treated (to predict probability that the specific disorder will be successfully treated).

As illustrated, the output from the machine learning model 410 (e.g., the output of the dynamic component 420, which is itself generated based on some or all of the input patient attributes 405A and/or condition data 405B and the output of the static component 415) is provided to an adjustment component 425. In some embodiments, this output may be referred to as an initial score, an efficacy score, an initial efficacy score, and the like. This initial score may generally indicate the predicted efficacy of the corresponding medication (either the specific medication or class of medications if the model is medication-specific, or the indicated medication if the model is medication-agnostic) in treating the corresponding disorder (either the specific disorder or class of disorders if the model is disorder-specific, or the indicated disorder if the model is disorder-agnostic), based on the specific patient's attributes.

However, as discussed above with reference to the static component 415, in some embodiments, particular features of the input data may be particularly predictive of the efficacy of the medication. In such an embodiment, the adjustment component 425 can receive these selected features (as illustrated) in the patient attributes 405A and/or condition data 405B and process them to generate an adjustment factor or score. This adjustment factor or score can then be used to modify the initial score, resulting in an overall efficacy score 430.

For example, in one such embodiment, the adjustment component 425 may determine whether the patient has already received or tried the corresponding medication without success. If so, the adjustment component 425 may generate an adjustment factor (e.g., a scaling factor) with a relatively low value, thereby reducing the initial score and outputting a relatively lower efficacy score 430. Similarly, in some embodiments, the adjustment component 425 may generate the adjustment factor based on data such as the current severity of the disorder (e.g., where higher severity results in an adjustment factor that reduces the predicted efficacy).

In some embodiments, use of such an adjustment component 425 after applying the machine learning model 410 can significantly improve the accuracy and operations of the model. For example, if the training data used to train the model is substantially unbalanced (e.g., with relatively few exemplars for a given class, such as an “adverse event” class), the adjustment component 425 may allow for more accurate efficacy scores by adjusting to such factors in ways that traditional trained models cannot. Similarly, if some attributes have particularly strong impacts on medication efficacy, the adjustment component 425 may be used to provide strong adjustments that a machine learning model may tend to ignore or smooth over.

In an embodiment, as discussed above, the efficacy score 430 can generally indicate the predicted efficacy of the corresponding medication with respect to the corresponding disorder and patient. In some embodiments, the efficacy score 430 includes a numerical value within a defined range (e.g., between zero and one). For example, the efficacy score 430 may be determined based on a predicted probability that the medication course will work to cure or treat the disorder (e.g., where the efficacy score is directly related to the probability of success), a predicted duration or time that will be required until the medication course works (e.g., where the efficacy score is inversely related to the duration), a magnitude of predicted improvement in the disorder (e.g., where the efficacy score is directly related to the predicted magnitude of improvement), a probability that the patient will suffer one or more adverse events (e.g., where the efficacy score is inversely related to the probability of adverse events), the predicted severity of any potential adverse events (e.g., where the efficacy score is inversely related to the predicted severity), and the like. In some embodiments, the efficacy score 430 can additionally or alternatively include other components, such as a classification (e.g., as a strong, moderate, or poor candidate medication course).

### Example Workflow for Preprocessing Unstructured Data for Improved Machine Learning

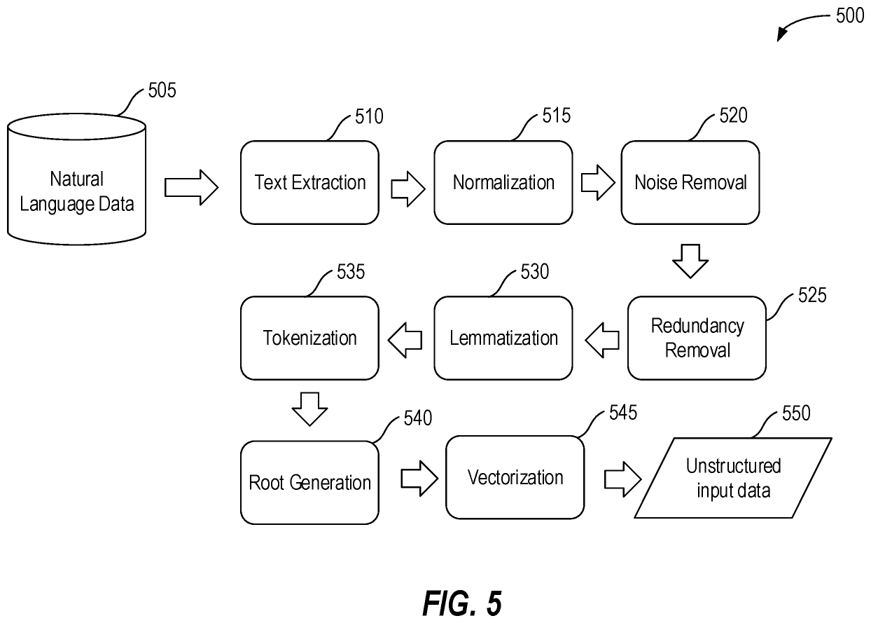

FIG. 5 depicts an example workflow 500 for preprocessing unstructured data for improved machine learning. In some embodiments, the workflow 500 may be performed to process natural language data 505 for input to one or more machine learning models. In some embodiments, the workflow 500 is performed by one or more remote systems (e.g., by a cloud-based service). In other embodiments, the workflow 500 is performed by a machine learning system, such as machine learning system 135 of FIG. 1, machine learning system 235 of FIG. 2, and/or machine learning system 335 of FIG. 3. The workflow 500 may be used during training of machine learning models (e.g., to generate the training input) and/or during inferencing using the models (e.g., as input to generate a predicted efficacy). That is, the workflow 500 may be used to transform or preprocess any natural language input, prior to it being used as input to the machine learning model(s) during training or inferencing.

In the illustrated workflow 500, natural language data 505 is received for processing to generate unstructured input data 550. In some embodiments, the workflow 500 is referred to as preprocessing to indicate that it is used to transform, refine, manage, or otherwise modify the natural language data 505 to improve its suitability for use with machine learning systems (or other downstream processing). In some embodiments, the natural language data 505 corresponds to progress notes, such as those authored by a clinician and indicating the progress or changes in patient condition (e.g., indicating whether a disorder is progressing, improving, unchanged, and the like).

In some embodiments, preprocessing the data in the natural language notes 505 may improve the ML training process by making the data more compatible with natural language processing, and ultimately for consumption by the ML model during training. Preprocessing can generally include a variety operations. Though the illustrated workflow 500 depicts a series of operations being performed sequentially for conceptual understanding, in embodiments, some or all of the operations may be performed in parallel. Similarly, in embodiments, the workflow 500 may include additional operations not depicted, or may include a subset of the depicted operations.

In the illustrated example, the natural language data 505 can first undergo text extraction 510. The text extraction 510 generally corresponds to extracting natural language text from an unstructured portion of the natural language data 505. For example, if the natural language data 505 includes a set of progress notes (e.g., notes written by a clinician describing an encounter with a user or patient), the text extraction 510 can include identifying and extracting these notes for evaluation. In some aspects, the notes may further include structured or semi-structured data that can undergo more traditional processing as needed, such as a timestamp indicating when the note was written or revised, an indication of the specific patient about whom the note was written, the author of the note, and the like.

The normalization 515 can generally a wide variety of text normalization processes, such as converting all characters in the extracted text to lowercase, converting accented or foreign language characters to ASCII characters, expanding contractions, converting words to numeric form where applicable, converting dates to a standard date format, and the like.

Noise removal 520 can generally include identification and removal of portions of the extracted text that do not carry meaningful or probative value. That is, noise removal 520 may include removing characters, portions, or elements of the text that are not useful or meaningful in the ultimate computing task (e.g., computing a predicted efficacy score), and/or that are not useful to human readers. For example, the noise removal 520 may include removing extra white or blank spaces, tabs, or lines, removing tags such as HTML tags, and the like.

Redundancy removal 525 may generally correspond to identifying and eliminating or removing text corresponding to redundant elements (e.g., duplicate words), and/or the reduction of a sentence or phrase to a portion thereof that is most suitable for machine learning training or application. For example, the redundancy removal 525 may include eliminating verbs (which may be unhelpful in the machine learning task), conjunctions, or other extraneous words that do not aid the machine learning task.

Lemmatization 530 can generally include stemming and/or lemmatization of one or more words in the extracted text. This may include converting words from their inflectional or other form to a base form. For example, lemmatization 530 may include replacing “holding,” “holds,” and “held” with the base form “hold.”

In one embodiment, tokenization 535 includes transforming or splitting elements in the extracted text (e.g., strings of characters) into smaller elements, also referred to as “tokens.” For example, the tokenization 535 may include tokenizing a paragraph into a set of sentences, tokenizing a sentence into a set of words, transforming a word into a set of characters, and the like. In some embodiments, tokenization 535 can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

In an embodiment, root generation 540 can include reducing portion of the extracted text (e.g., a phrase or sentence) to its most relevant n-gram (e.g., a bigram) or root for downstream machine learning training and/or application.

Vectorization 545 may generally include converting the text into one or more objects that can be represented numerically (e.g., into a vector or tensor form). For example, the vectorization 545 may use one-hot encodings (e.g., where each element in the vector indicates the presence or absence of a given word, phrase, sentiment, or other concept, based on the value of the element). In some embodiments, the vectorization 545 can correspond to any word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate efficacy scores).

As illustrated, the various preprocessing operations in the workflow 500 result in generation of unstructured input data 550. That is, the unstructured input data 550 corresponds to unstructured natural language data 505 that has undergone various preprocessing to improve its use with downstream machine learning models. The preprocessing workflow 500 can generally include any other suitable techniques for making text ingestion more efficient or accurate (either in a training phase of a machine learning model, or while generating an inference or prediction using a trained model). Generally, improving the results of this natural language processing can have significant positive impacts on the computational efficiency of processing the data downstream, as well as the eventual accuracy of the trained machine learning model(s).

In some embodiments, as discussed above, this unstructured input data 550 is provided to a machine learning model, such as to the static portion of a hybrid model, to the dynamic portion of a hybrid model, and/or to the adjustment portion of a hybrid model.

### Example Method for Training Machine Learning Models to Predict Efficacy

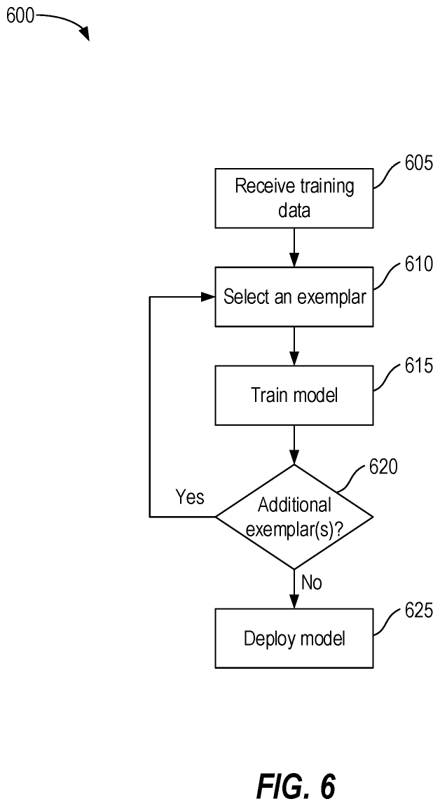

FIG. 6 is a flow diagram depicting an example method 600 for training machine learning models to predict efficacy. In the illustrated embodiment, the method 600 is performed by a machine learning system, such as the machine learning system 135 of FIG. 1, the machine learning system 235 of FIG. 2, and/or the machine learning system 335 of FIG. 3. In other embodiments, the method 600 can be performed by other systems, such as dedicated training systems.

The method 600 begins at block 605, where the machine learning system receives training data (e.g., historical data 105 of FIG. 1) to train one or more machine learning models. In embodiments, receiving the training data may include receiving it from one or more other systems (e.g., data aggregation or preprocessing systems), retrieving it from local or remote storage, and the like. In an embodiment, the training data can generally include a set of exemplars, where each exemplar includes a set of patient attributes (including information such as the patient's demographics, allergies, current medications, diagnoses or conditions, and the like) and a label indicating the results achieved using a corresponding medication plan. For example, a first exemplar may indicate that the patient is a 65 year old male that is allergic to penicillin and has a minor sinus infection, and is being treated with a specific course of antibiotics (where the course can specify the class of drug, a specific drug, a dosage (e.g., amount of medication per pill or other consumption format), a timing (e.g., twice a day), a duration (e.g., for two weeks), and the like). The label associated with the exemplar may indicate whether the sinus infection was successfully treated (and if so, how long it took for the infection to be mitigated or remediated), whether any adverse events occurred (e.g., whether the antibiotics caused any issues such as nausea), the onset of such adverse conditions, and the like.

In embodiments, the training data may include exemplars corresponding to any number and variety of patients, disorders, and medication courses. In some embodiments, if the training data includes data for multiple conditions and/or medications, the machine learning system can use it to train a model to predict efficacy for multiple conditions and/or medications. In at least one embodiment, as discussed above, the machine learning system may train disorder-specific models (e.g., models trained to predict medicine efficacy for a single disorder or class of disorders using any medication), medication-specific models (e.g., models trained to predict efficacy of various courses of a specific medication or class of medications for any disorder), disorder-specific and medication-specific models (e.g., models trained to predict the efficacy of various courses of a specific medication or class of medications for treating a specific disorder or class of disorders), and the like. In an embodiment, if the machine learning system is training such a specific model, the training data may include only exemplars corresponding to that class (e.g., to the specific medication and/or disorder). In other embodiments, the machine learning system may select the appropriate records from the training data (e.g., only using the appropriate records to train the model).

At block 610, the machine learning system selects one of the exemplars included in the training data. As discussed above, an exemplar refers to a set of attributes (e.g., which may include a defined or learned set of features) from a single patient and associated with a particular condition or diagnosis. For example, the attributes may correspond to those present during a defined point or window of time when the condition was present (e.g., at the time of diagnosis or initiation of treatment, within a period before the diagnosis or initiation of treatment, during treatment, and the like). As an example, the exemplar may include indications as to the demographics of the patient, whether the patient had any other specified diagnoses at the time, whether the patient used any other medication(s) at the time, allergies of the user, and the like. In some embodiments, the exemplar further indicates the medication plan that was used to treat the condition/diagnosis.

Generally, the exemplar may be selected using any suitable criteria (including randomly or pseudo-randomly), as the machine learning system will use all exemplars during the training process. Although the illustrated example depicts selecting exemplars sequentially for conceptual clarity, in embodiments, the machine learning system may select and/or process multiple exemplars in parallel.

At block 615, the machine learning system trains a machine learning model based on the selected exemplar. For example, the machine learning system may use the attributes and medication plan indicated in the exemplar to generate an output efficacy score or classification for the medication plan, with respect to the patient attributes. As discussed above, this efficacy score can generally indicate the predicted efficacy of the medication plan, such as the probability that the medication plan will allow or cause the patient to recover from the condition (or otherwise reach the relevant goal(s) of the plan, such as halting further decline or maintaining the current state), a predicted time or duration that will pass before the recovery occurs (or until the goal(s) are reached), and the like. In one such embodiment, lower values may indicate a lower probability that the patient will reach the goals, while a higher value indicates that the patient is relatively more likely to reach the indicated goal(s).

During training, this score can then be compared against a ground-truth associated with the selected exemplar (e.g., an indication as to whether the resident did, in fact, recover or otherwise reach the goal(s), or whether any adverse events occurred). In some embodiments, this comparison includes determining how much time elapsed between the start of the medication plan and the eventual recovery (or time when adverse event(s) occurred, or when the plan was terminated). Based on this comparison, the parameters of the machine learning model can be updated. For example, if the generated recovery score is relatively low but the resident did, in fact, recover completely, the machine learning system may modify the parameters such that the attributes and medication plan in the exemplar result in a larger efficacy score being generated. Similarly, if the predicted score is relatively high but the patient actually took a significant time to recover (e.g., beyond the indicated duration of the medication plan), the machine learning system may learn to associate a lower efficacy score with these attributes.

In some embodiments, as discussed above, the machine learning system uses a hybrid model including one or more static portions and one or more dynamic portions. In some embodiments, the machine learning system can also use an adjustment portion to further improve the resulting predictions. Additionally, in some embodiments, as discussed above, the machine learning system may train the model to receive the medication plan as input (e.g., for medication-agnostic models), or to receive only specified portions (such as the dosage and duration), such as for medication specific-models.

In some embodiments, as discussed above, the system can additionally or alternatively use a purely dynamic model (e.g., with no static portion) or a purely static model (e.g., with no dynamic or trained portion).

At block 620, the machine learning system determines whether at least one additional exemplar remains in the training data. If so, the method 600 returns to block 610. If not, the method 600 continues to block 625. Although the illustrated example depicts iteratively refining the model using individual exemplars (e.g., using stochastic gradient descent), in some embodiments, the machine learning system can refine the model based on multiple exemplars simultaneously (e.g., using batch gradient descent).

At block 625, the machine learning system deploys the trained machine learning model for runtime use. In embodiments, this may include deploying the model locally (e.g., for runtime use by the machine learning system) and/or to one or more remote systems. For example, the machine learning system may distribute the trained model to one or more downstream systems (e.g., associated with care settings, such as clinics and hospitals).

### Example Method for Predicting Efficacy Using Machine Learning

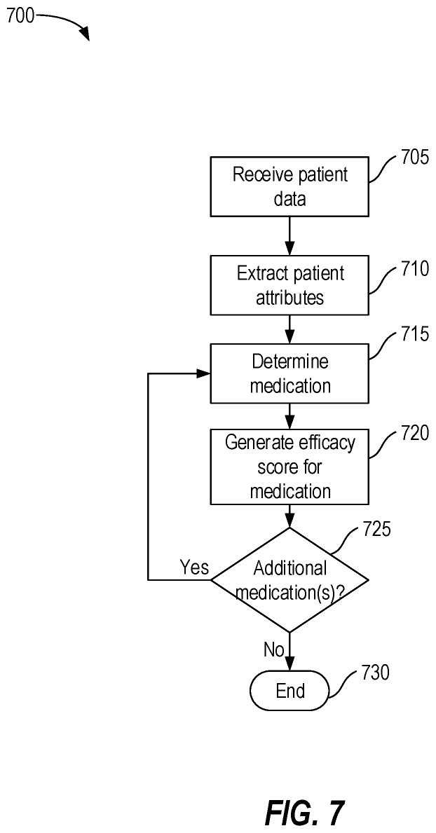

FIG. 7 is a flow diagram depicting an example method 700 for predicting efficacy using machine learning models. In the illustrated embodiment, the method 700 is performed by a machine learning system, such as the machine learning system 135 of FIG. 1, the machine learning system 235 of FIG. 2, and/or the machine learning system 335 of FIG. 3. In other embodiments, the method 700 can be performed by other systems, such as dedicated inferencing systems.

At block 705, the machine learning system receives patient data (e.g., patient data 205 of FIG. 2 and/or patient data 305 of FIG. 3). As discussed above, the patient data may include information associated with a patient (also referred to as a user or a resident in some aspects).

In an embodiment, the patient data can generally include information relating to attributes of the patient, such as one or more demographics of the patient (e.g., age, race, sex, and the like), a list of allergies of the patient, a list of any diagnoses or conditions of the patient, medications used by the patient, assistance needed by the patient (e.g., help with walking), and the like. In some embodiments, the received patient data corresponds to current information for the patient. That is, the patient data may be the most-recent data for each feature.

In at least one aspect, the patient data is received because a change has occurred. That is, the patient data may be provided to the machine learning system (e.g., using a push technique) based on determining that one or more of the attributes have changed since the last time the data was provided. In other embodiments, the machine learning system can retrieve or request the patient data, and evaluate it to determine whether any attributes have changed. In at least one embodiment, if no attributes have changed (with respect to the relevant features used by the model), the machine learning system can refrain from further processing of the data (e.g., refrain from generating a new care plan), thereby reducing computational expense.

Similarly, if the data is only provided upon detecting a change, the machine learning system need not review it at all, which also reduces computational expense of the system. Additionally, in some embodiments, the machine learning system can receive only the updated data (as opposed to receiving or retrieving the entirety of the patient data). That is, the storage systems may automatically transmit data when it is updated (or the machine learning system may request any new or changed data), enabling medication plans to be evaluated and/or revised based on the new data without the need to re-transmit the older data. This, again, reduces the computational expense (including bandwidth, if the data is stored remotely from the machine learning system) of generating the scores.

In some embodiments, the received patient data includes data corresponding to a defined set of features that are used by the machine learning model. These features may include, for example, specific demographic data, specified diagnoses, specified medications, and the like. In some embodiments, the patient data can include additional data beyond these features (e.g., information about all medications that the patient has been prescribed, regardless of the specific medications used in the machine learning model). In one such embodiment, the machine learning system can identify and extract the relevant attributes or data, based on the indicated features for the model. In other embodiments, the received patient data may include only the specific data corresponding to the indicated features (e.g., another system may filter the patient data based on the features, thereby protecting data that is not used by the model). In still another aspect, such unused features or attributes may simply be associated with a weight of zero in the model.

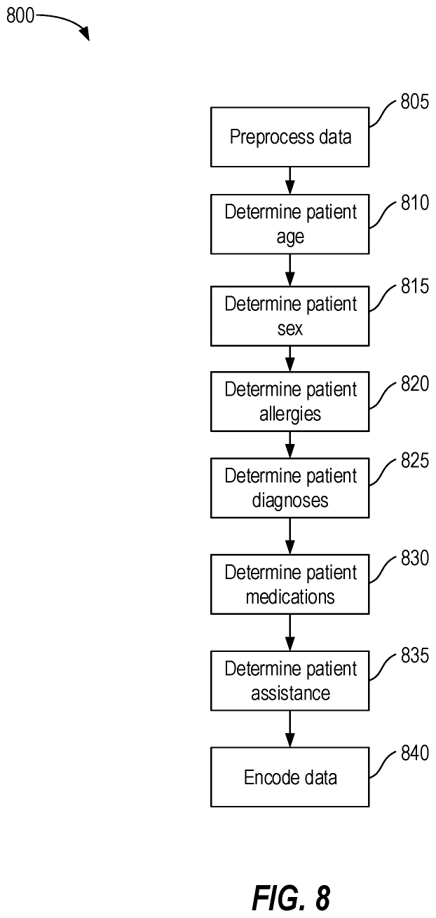

At block 710, the machine learning system extracts the set of relevant patient attributes, from the patient data, based on the specified features that are used by the machine learning model. That is, the machine learning system can extract, from the patient data, the relevant information for each feature. For example, if a specific demographic feature (e.g., age) is included in the features, the machine learning system may search the patient data to identify and extract the patient age. In some embodiments, extracting the patient attributes can include further preprocessing, such as using natural language processing techniques to evaluate written progress notes. One example of extracting patient attributes is described in more detail below with reference to FIG. 8.

At block 715, the machine learning system determines the medication course that is being evaluated for the patient. In some embodiments, this includes identifying a medication plan specified or suggested by a clinician (or selecting one of the possible alternatives suggested). In some embodiments, this includes identifying possible treatments based on the indicated diagnosis (e.g., reviewing published literature to identify specific medications and/or classes of medication that can be used to treat the indicated diagnosis or disorder). Generally, if multiple medication(s) or medication plans are indicated or identified, selecting among them can be performed using any technique (including randomly or pseudo-randomly), as the machine learning system will evaluate all of the alternatives during the method 700. Although an iterative or sequential process is depicted for conceptual clarity, in some embodiments, the machine learning system may evaluate some or all of the medication plans in parallel.

At block 720, the machine learning system processes the identified/extracted attributes using a trained machine learning model to generate an efficacy plan indicating the probability that the patient will recover using the selected plan. As discussed above, the machine learning model may generally specify a set of parameters (e.g., weights) for input features and/or intermediate features (within internal portions of the model) learned during training.

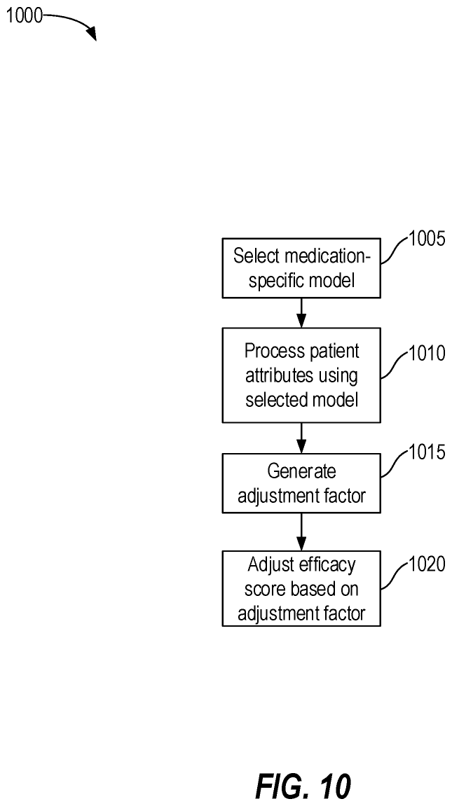

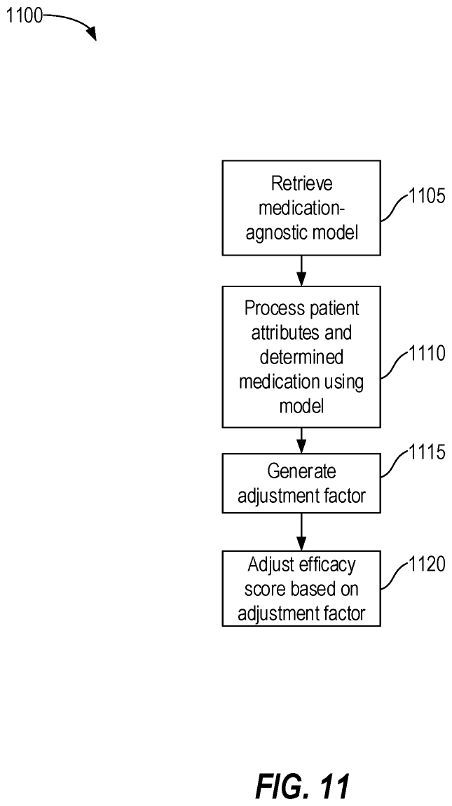

In some embodiments, as discussed above and in more detail below with reference to FIG. 10, the machine learning system may generate the efficacy score by using a medication-specific model that was trained to predict efficacies for the selected medication (or class of medications). In some embodiments, as discussed above and in more detail below with reference to FIG. 11, the machine learning system may use a medication-agnostic model that can generate efficacies for a variety of medications.

Additionally, in some embodiments, as discussed above, the system can additionally or alternatively use a purely dynamic model (e.g., with no static portion) to generate the efficacy score, or may use a purely static model (e.g., with no dynamic or trained portion) to generate the efficacy score.

At block 725, the machine learning system determines whether there is at least one alternative medication plan that has not-yet been evaluated for the patient. If so, the method 700 returns to block 715. If not, the method 700 terminates at block 730.

In some embodiments, the machine learning system can optionally implements the medication plan(s), output the medication plan(s) and efficacy score(s) to a user for approval, and the like.

### Example Method for Extracting and Preprocessing Data for Machine Learning

FIG. 8 is a flow diagram depicting an example method 800 for extracting and preprocessing patient data for improved machine learning. In the illustrated embodiment, the method 800 is performed by a machine learning system, such as the machine learning system 135 of FIG. 1, the machine learning system 235 of FIG. 2, and/or the machine learning system 335 of FIG. 3. In other embodiments, the method 800 can be performed by other systems, such as dedicated inferencing systems.

In embodiments, the method 800 may be performed during a training phase for a machine learning model (e.g., to generate training data, or to process exemplars during a forward-pass of the training phase). Similarly, in embodiments, the method 800 may be used during an inference phase (e.g., to extract and preprocess input data during runtime).

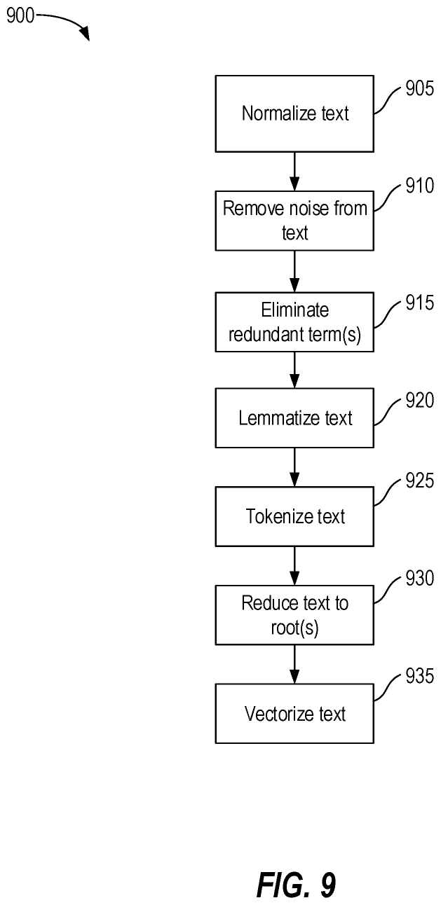

At block 805, the machine learning system optionally preprocesses the patient data. In embodiments, this preprocessing can include a wide variety of operations and techniques, depending on the underlying patient data and the needs of the specific machine learning architecture. For example, if the data includes natural language text, preprocessing may include operations such as normalization and keyword identification, as discussed in more detail below with reference to FIG. 9.

At block 810, the machine learning system determines the age of the patient, as indicated in the received data. At block 815, the machine learning system can determine the biological sex of the patient. Further, at block 820, the machine learning system determines any allergies of the patient (or a set of defined allergies, such as being allergic to specific medications).

At block 825, the machine learning system determines a set of diagnoses of the patient (which may include determining any diagnoses, or determining whether the patient has any predefined diagnoses). In some embodiments, at block 825, the machine learning system also determines which diagnosis (or diagnoses) is currently being treated. That is, the machine learning system can determine which diagnosis should be evaluated (e.g., as indicated by a physician) to generate an efficacy score.

At block 830, the machine learning system determines a list of medications that the patient currently uses (which may include determining any medications, or determining whether the patient uses any predefined medications). In some embodiments, at block 830, the machine learning system also determines which medication plan (or plans) are currently being evaluated. That is, the machine learning system can determine which medication plan(s) should be evaluated (e.g., as indicated by a physician) to generate an efficacy score.

At block 835, the machine learning system determines any assistance needed by the patient (or whether the patient needs any of a defined set of assistances). For example, if the patient is a resident of a long-term care facility, the machine learning system may determine whether the patient needs assistance with standing, sitting, walking, transferring, eating, and the like.

Although the illustrated method 800 depicts specific data components (such as age and allergies) for conceptual clarity, in embodiments, it is to be understood that the machine learning system may use any variety of data, including information not depicted in the illustrated example (such as the patient's race), and may further refrain from considering some or all of the indicated information (e.g., refrain from considering assistances needed).

At block 840, the machine learning system optionally encodes the data to suit the machine learning architecture. For example, as discussed above, the machine learning system may generate numerical vectors representing the determined data (e.g., using one-hot encoding).

The data can then be processed by one or more machine learning models (which may include hybrid models) to train the model (e.g., by comparing a predicted efficacy with a known ground-truth efficacy) and/or to generate or predict medication efficacy during runtime, as discussed above.

### Example Method for Preprocessing Unstructured Data to Improve Machine Learning

FIG. 9 is a flow diagram depicting an example method 900 for preprocessing unstructured input data to improve machine learning results. In the illustrated embodiment, the method 900 is performed by a machine learning system, such as the machine learning system 135 of FIG. 1, the machine learning system 235 of FIG. 2, and/or the machine learning system 335 of FIG. 3. In other embodiments, the method 900 can be performed by other systems, such as dedicated natural language processing systems or preprocessing systems. In some embodiments, the method 900 provides additional detail for the workflow 500 of FIG. 5, and/or for block 805 in FIG. 8.

Generally, each block in the method 900 is optional, and the machine learning system may perform all of the indicated operations, or some subset thereof. The machine learning system may also use additional preprocessing steps not depicted in the illustrated example. Additionally, though the illustrated example suggests a linear and sequential process for conceptual clarity, in embodiments, the operations may be performed in any order (including entirely or partially in parallel).

In an embodiment, the method 900 can be used to preprocess natural language text extracted from written notes, such as progress notes authored by a physician. For example, a healthcare provider may write notes relating to a patient's progress, such as whether a disease is progressing, improving, or stagnant, whether any adverse events or concerns (such as nausea) were reported by the patient, and the like. This extracted text can be processed to glean insights useful for predicting medication efficacy (e.g., as input to a static portion of a hybrid model), but may require some level of preprocessing first in some aspects.

At block 905, the machine learning system can normalize the extracted natural language text. As discussed above, this normalization may include a wide variety of text normalization processes, such as converting all characters in the extracted text to lowercase, converting accented or foreign language characters to ASCII characters, expanding contractions, converting words to numeric form where applicable, converting dates to a standard date format, and the like.

At block 910, the machine learning system removes noise from the text. As discussed above, noise removal may include identification and removal of portions of the extracted text that do not carry meaningful or probative value, such as characters, portions, or elements of the text that are not useful or meaningful in the ultimate computing task (e.g., predicting medication efficacy), and/or that are not useful to human readers. For example, the noise removal may include removing extra white or blank spaces, tabs, or lines, removing tags such as HTML tags, and the like.

At block 915, the machine learning system can eliminate redundant elements or terms from the text. As discussed above, this may include identifying and eliminating or removing text corresponding to redundant elements (e.g., duplicate words), and/or the reduction of a sentence or phrase to a portion thereof that is most suitable for machine learning training or application. For example, the redundancy elimination may include eliminating verbs (which may be unhelpful in the machine learning task), conjunctions, or other extraneous words that do not aid the machine learning task.

At block 920, the machine learning system lemmatizes the text. As discussed above, text lemmatization can generally include stemming and/or lemmatization of one or more words in the extracted text. This may include converting words from their inflectional or other form to a base form. For example, lemmatization may include replacing “holding,” “holds,” and “held” with the base form “hold.”

At block 925, the machine learning system tokenizes the text. In an embodiment, tokenizing the text may include transforming or splitting elements in the extracted text (e.g., strings of characters) into smaller elements, also referred to as “tokens.” For example, the tokenization may include tokenizing a paragraph into a set of sentences, tokenizing a sentence into a set of words, transforming a word into a set of characters, and the like. In some embodiments, tokenization can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

At block 930, the machine learning system can reduce the text to one or more roots. As discussed above, the root generation can include reducing portion of the extracted text (e.g., a phrase or sentence) to its most relevant n-gram (e.g., a bigram) or root for downstream machine learning training and/or application.

At block 935, the machine learning system can vectorize the text. Generally, vectorization may include converting the text into one or more objects that can be represented numerically (e.g., into a vector or tensor form). For example, the machine learning system may use one-hot encodings (e.g., where each element in the vector indicates the presence or absence of a given keyword, phrase, sentiment, or other concept, based on the value of the element). In some embodiments, the machine learning system can generate one or more word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate efficacy scores).

For example, in some embodiments, the preprocessed data can be used as input to a machine learning model or other NLP techniques (such as keyword matching) that predicts whether it indicates improving or worsening disease, that classifies the severity of the disease, that identifies or classifies severity of adverse events, and the like. In some embodiments, this processing can be performed as part of a static component of a hybrid machine learning model, and the resulting data (e.g., scores indicating the probability that the patient has particular adverse events) can be used as input to the dynamic portion of the hybrid model.

### Example Method for Generating Predicted Efficacy Using Specific Machine Learning Models

FIG. 10 is a flow diagram depicting an example method 1000 for generating efficacy scores using medication-specific machine learning models. In the illustrated embodiment, the method 1000 is performed by a machine learning system, such as the machine learning system 135 of FIG. 1, the machine learning system 235 of FIG. 2, and/or the machine learning system 335 of FIG. 3. In other embodiments, the method 1000 can be performed by other systems, such as dedicated inferencing systems. In some embodiments, the method 1000 provides additional detail for block 720 in FIG. 7.

At block 1005, the machine learning system selects a medication-specific model that has been trained for the medication (or class of medications) that is being evaluated for the patient. That is, the machine learning system can identify, retrieve, load, or otherwise select a machine learning model (which may be a hybrid model, as discussed above) that was trained using data associated with the medication (or class of medication) that is being tested. In some embodiments, the model is trained to receive patient attributes as input, and generate a predicted efficacy for the corresponding medication as output.

At block 1010, the machine learning system processes the patient attributes using the selected model. For example, the machine learning system may process the patient's demographics, other medications, diagnoses, and the like. In some embodiments, some or all of the attributes may be preprocessed and/or encoded, as discussed above. In at least one embodiment, the model further receives one or more aspects of the medication course, such as the dosage, the timing, the duration, and the like. That is, the model may be trained to predict efficacy of a given medication (or class of medications) based on patient attributes, and based further on the specific course or plan. In this way, the model can be used to predict efficacy stores based not only on the underlying patient data, but also on the selected dosage, duration, and the like.

In some embodiments, as discussed above, the machine learning model is a hybrid model. In one such embodiment, the machine learning system can process a defined subset of attributes using the static portion of the model (e.g., processing natural language text to generate scores for progress notes). The output of the static portion (e.g., one or more scores or vectors representing the subset of attributes) can then be provided to the dynamic portion of the model (which may also receive other patient attributes directly).

Additionally, in some embodiments, as discussed above, the system can additionally or alternatively use a purely dynamic model (e.g., with no static portion) to process the patient attributes, or may use a purely static model (e.g., with no dynamic or trained portion) to process the patient attributes.

In an embodiment, as discussed above, processing the data using the model results in the generation of a score indicating the predicted efficacy of the medication course for the specific patient. In some embodiments, as discussed above, this score may be referred to as an initial score.

At block 1015, the machine learning system can optionally generate an adjustment factor, based on the input data, to modify the initial efficacy score output by the model. For example, as discussed above a defined subset of the input patient attributes may be used to generate an adjustment factor reflecting how likely the medication is to work (or how likely it is to result in adverse events). For example, in one such embodiment, the adjustment factor is generated based on determining whether the patient already tried the indicated medication (or a related medication, such as one in the same class), whether the patient previously had an adverse event using the medication (or a related medication), the current and/or historical severity of the disorder, and the like.

In some embodiments, the adjustment factor is used as a scaling value that increases or decreases the initial efficacy score (e.g., by multiplying the initial score by the adjustment factor, by adding or subtracting the adjustment factor, by averaging the initial score and the adjustment factor, and the like).

At block 1020, the machine learning system then adjusts the efficacy score based on the generated adjustment factor, resulting in a final predicted efficacy. This predicted efficacy can be used to drive improved treatment selections. For example, the efficacy may be output (e.g., via a GUI) to a user (such as a physician), allowing the user to determine whether the medication course should be used. In some embodiments, the efficacy score can be used to sort and/or filter alternative medication plans, allowing the user to easily identify the most effective options.

### Example Method for Generating Predicted Efficacy Using Agnostic Machine Learning Models

FIG. 11 is a flow diagram depicting an example method 1100 for generating efficacy scores using medication-agnostic machine learning models. In the illustrated embodiment, the method 1100 is performed by a machine learning system, such as the machine learning system 135 of FIG. 1, the machine learning system 235 of FIG. 2, and/or the machine learning system 335 of FIG. 3. In other embodiments, the method 1100 can be performed by other systems, such as dedicated inferencing systems. In some embodiments, the method 1100 provides additional detail for block 720 in FIG. 7.

At block 1105, the machine learning system selects a medication-agnostic model that has been trained to predict efficacy scores for a variety of medications. That is, the machine learning system can identify, retrieve, load, or otherwise select a machine learning model (which may be a hybrid model, as discussed above) that was trained using data associated with any number and variety of medications (or classes of medication) that is being tested. In some embodiments, the model is trained to receive patient attributes (including the medication course being used) as input, and generate a predicted efficacy for the indicated medication course as output.

At block 1110, the machine learning system processes the patient attributes and the indicated medication course using the selected model. For example, the machine learning system may process the patient's demographics, other medications, diagnoses, and the like, along with the specific details of the indicated plan (e.g., the medication class, dosage, duration, and the like). In some embodiments, some or all of the attributes may be preprocessed and/or encoded, as discussed above. In this way, the model can be used to predict efficacy stores based not only on the underlying patient data, but also on the specific medication, dosage, duration, and the like.

In some embodiment, as discussed above, the machine learning model is a hybrid model. In one such embodiment, the machine learning system can process a defined subset of attributes using the static portion of the model (e.g., processing natural language text to generate scores for progress notes). The output of the static portion (e.g., one or more scores or vectors representing the subset of attributes) can then be provided to the dynamic portion of the model (which may also receive other patient attributes directly).

Additionally, in some embodiments, as discussed above, the system can additionally or alternatively use a purely dynamic model (e.g., with no static portion) to process the patient attributes, or may use a purely static model (e.g., with no dynamic or trained portion) to process the patient attributes.

In an embodiment, as discussed above, processing the data using the model results in the generation of a score indicating the predicted efficacy of the indicated medication course for the specific patient. In some embodiments, as discussed above, this score may be referred to as an initial score.

At block 1115, the machine learning system can optionally generate an adjustment factor, based on the input data, to modify the initial efficacy score output by the model. For example, as discussed above a defined subset of the input patient attributes may be used to generate an adjustment factor reflecting how likely the medication is to work (or how likely it is to result in adverse events). For example, in one such embodiment, the adjustment factor is generated based on determining whether the patient already tried the indicated medication (or a related medication, such as one in the same class), whether the patient previously had an adverse event using the medication (or a related medication), the current and/or historical severity of the disorder, and the like.

In some embodiments, the adjustment factor is used as a scaling value that increases or decreases the initial efficacy score (e.g., by multiplying the initial score by the adjustment factor, by adding or subtracting the adjustment factor, by averaging the initial score and the adjustment factor, and the like).

At block 1120, the machine learning system then adjusts the efficacy score based on the generated adjustment factor, resulting in a final predicted efficacy. This predicted efficacy can be used to drive improved treatment selections. For example, the efficacy may be output (e.g., via a GUI) to a user (such as a physician), allowing the user to determine whether the medication course should be used. In some embodiments, the efficacy score can be used to sort and/or filter alternative medication plans, allowing the user to easily identify the most effective options.

### Example Method for Refining Machine Learning Models

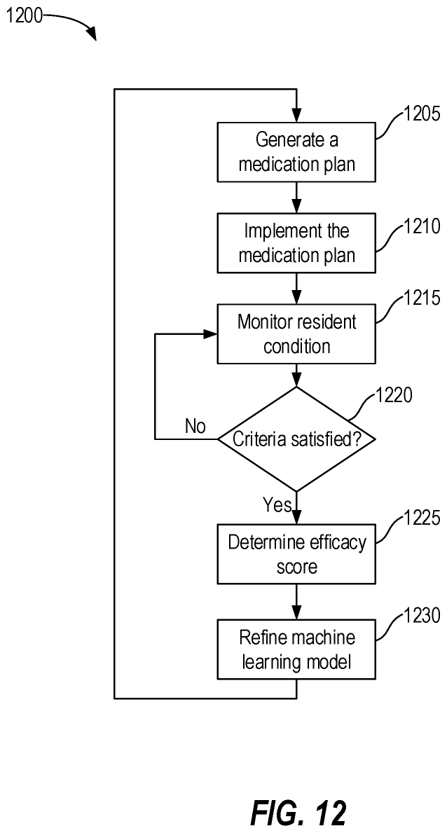

FIG. 12 is a flow diagram depicting an example method 1200 for refining machine learning models to generate efficacy scores. In the illustrated embodiment, the method 1200 is performed by a machine learning system, such as the machine learning system 135 of FIG. 1, the machine learning system 235 of FIG. 2, and/or the machine learning system 335 of FIG. 3. In other embodiments, the method 1200 can be performed by other systems, such as dedicated inferencing, training, or refinement systems. In some embodiments, the method 1200 provides additional detail for the workflow 300 of FIG. 3.

At block 1205, the machine learning system can generate one or more medication plans for a patient during runtime. That is, the machine learning system may deploy and use a trained machine learning model to process patient data in order to generate predicted efficacy scores for one or more medication plans at runtime, and select one or more of these medications plans for use. For example, the machine learning system may automatically use the model(s) to generate medication scores for a set of possible or alternative medication plans (e.g., as indicated by the physician, or by reviewing the medical literature). Similarly, the machine learning system may automatically generate new scores whenever new data becomes available. In some embodiments, a clinician can selectively use the machine learning system to generate efficacy scores and plans as needed.

At block 1210, the machine learning system implements the medication plan. In some embodiments, implementing the medication plan can include outputting it to a user (e.g., a clinician) for review and approval. Once the medication plan is in place, the patient begins receiving and using the indicated medication according to the plan.

At block 1215, the machine learning system (or another system, such as the monitoring system 350 of FIG. 3) monitors the patient condition(s) to determine whether any changes occur (such as an improvement in the condition, a worsening of the condition, complete remediation of the condition, and the like). In some embodiments, monitoring includes evaluating progress notes as discussed above, or otherwise monitoring for indications that the medication plan is being discontinued.

At block 1220, the machine learning system determines whether one or more defined criteria are satisfied. In some embodiments, the criteria can include an indication that the patient condition as changed (such as determined based on the monitoring of block 1215 above). In some embodiments, the criteria may include explicit feedback from a clinician (e.g., indicating that the medication plan is inadequate or inappropriate, indicating that the patient is no longer following the care plan for other reasons, and the like).

In at least one embodiment, the criteria can include determining whether a defined period of time has elapsed. In one such embodiment, the machine learning system can determine the duration and/or target date(s) specified in the medication plan. In such an embodiment, the criteria may include determining whether the current date is equal to or later than one or more target date(s), or whether the indicated duration has elapsed.

If, at block 1220, the machine learning system determines that none of the criteria have been satisfied, the method 1200 returns to block 1215. If at least one of the criteria is satisfied, the method 1200 continues to block 1225, where the machine learning system determines an efficacy score for the medication plan based on the patient's current condition. In one embodiment, the machine learning system determines the efficacy score by evaluating the patient attributes to determine whether the condition improved, worsened, or ceased. Generally, determining that the condition has terminated will result in a higher efficacy score as compared to a determination that the condition has improved but not-yet ended. Similarly, a determination that the condition improved is scored higher than a determination that the condition is unchanged, which is in turn scored higher than a determination that the condition has worsened.

In some embodiments, the efficacy score is also determined at least in part on the magnitude of the change (e.g., whether the patient is 20% improved or 50% improved), where larger improvements are associated with higher scores (and larger declines are associated with smaller scores). In at least one embodiment, the recovery score is determined based in part on the length of time that has elapsed since the medication plan was begun (e.g., where shorter times may be directly related to higher scores). In some embodiments, the efficacy score is based on the length of time that has elapsed, as compared to the target date(s) or duration of the plan. For example, a medication plan may receive a high recovery score if the condition was completely ameliorated, even if it took one year, if the plan was projected to take a year or more (as indicated by the duration). In some embodiments, the efficacy score is determined based on whether any adverse events occurred, how much time elapsed before the adverse event(s), and the like.

Once the recovery score for the relevant care plan (or portions thereof) has been generated, the method 1200 continues to block 1230. At block 1230, the machine learning system refines the trained machine learning model(s) based on this updated efficacy score. For example, if the generated efficacy score for the patient was relatively low, but the new data indicates that the patient condition has significantly improved, the machine learning system may use the prior set of attributes (used to generate the original score) as exemplar input that should be correlated with a high efficacy score. Similarly, if the generated score was relatively high but the new data indicates the patient condition has significantly worsened, the machine learning system may refine the model to indicate that such attributes should be correlated to a lower efficacy score.

The method 1200 then returns to block 1205. In this way, the machine learning system can continuously or periodically refine the machine learning model(s), thereby ensuring that they continue to produce highly-accurate efficacy score predictions.

### Example Method for Predicting Treatment Efficacy Using Machine Learning

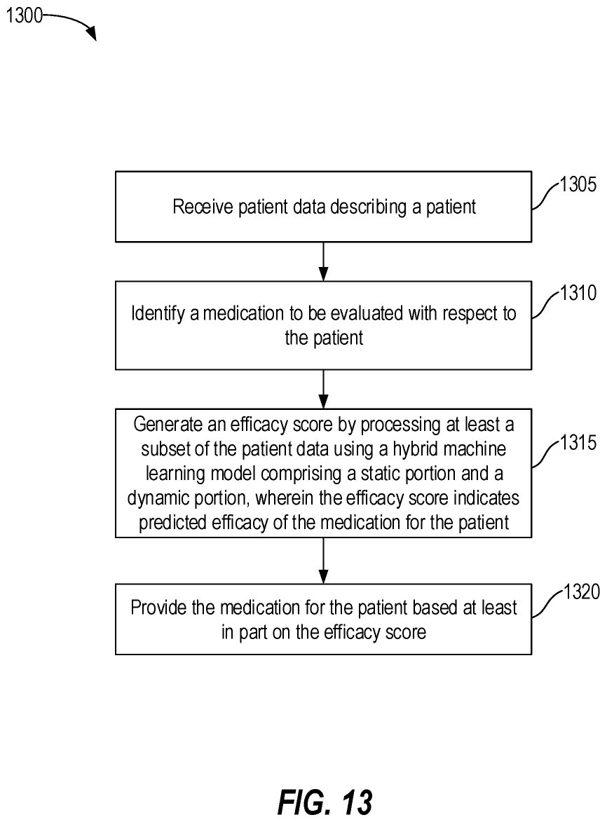

FIG. 13 is a flow diagram depicting an example method 1300 for generating efficacy scores using trained machine learning models. In the illustrated embodiment, the method 1300 is performed by a machine learning system, such as the machine learning system 135 of FIG. 1, the machine learning system 235 of FIG. 2, and/or the machine learning system 335 of FIG. 3. In other embodiments, the method 1300 can be performed by other systems, such as dedicated inferencing systems.

At block 1305, patient data (e.g., patient data 205 of FIG. 2) describing a patient is received.

At block 1310, a medication to be evaluated with respect to the patient (e.g., medication plan 240 of FIG. 2) is identified.

At block 1315, an efficacy score (e.g., efficacy score 245 of FIG. 2) is generated by processing at least a subset of the patient data using a hybrid machine learning model comprising a static portion and a dynamic portion, wherein the efficacy score indicates predicted efficacy of the medication for the patient.

At block 1320, the medication is provided for the patient based at least in part on the efficacy score.

### Example Method for Training Machine Learning Models to Predict Treatment Efficacy

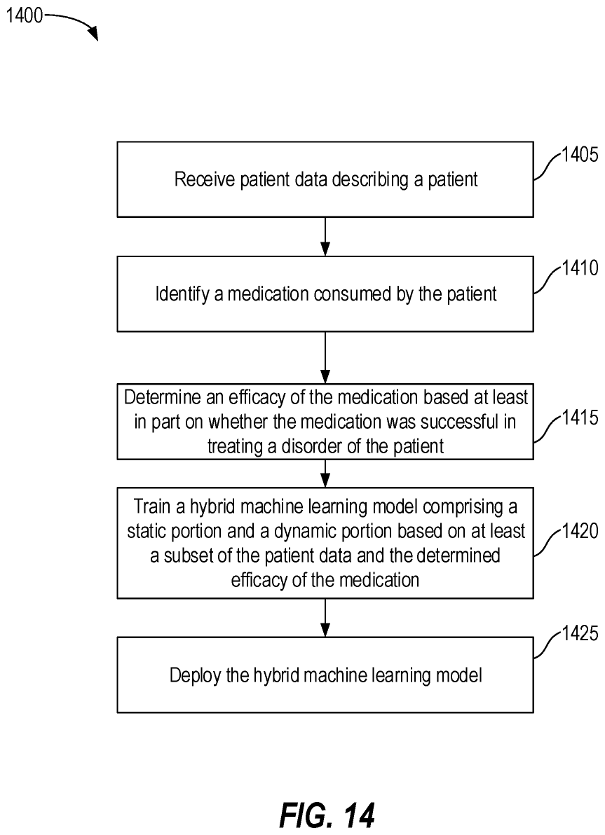

FIG. 14 is a flow diagram depicting an example method for training machine learning models to predict efficacy. In the illustrated embodiment, the method 1400 is performed by a machine learning system, such as the machine learning system 135 of FIG. 1, the machine learning system 235 of FIG. 2, and/or the machine learning system 335 of FIG. 3

At block 1405, patient data (e.g., historical data 105 of FIG. 1) describing a patient is received.

At block 1410, a medication consumed by the patient (e.g., medications 130 of FIG. 1) is identified.

At block 1415, an efficacy of the medication is determined based at least in part on whether the medication was successful in treating a disorder of the patient.

At block 1420, a hybrid machine learning model (e.g., machine learning model 140 of FIG. 1) comprising a static portion and a dynamic portion is trained based on at least a subset of the patient data and the determined efficacy of the medication.

At block 1425, the hybrid machine learning model is deployed.

### Example Processing System for Improved Machine Learning

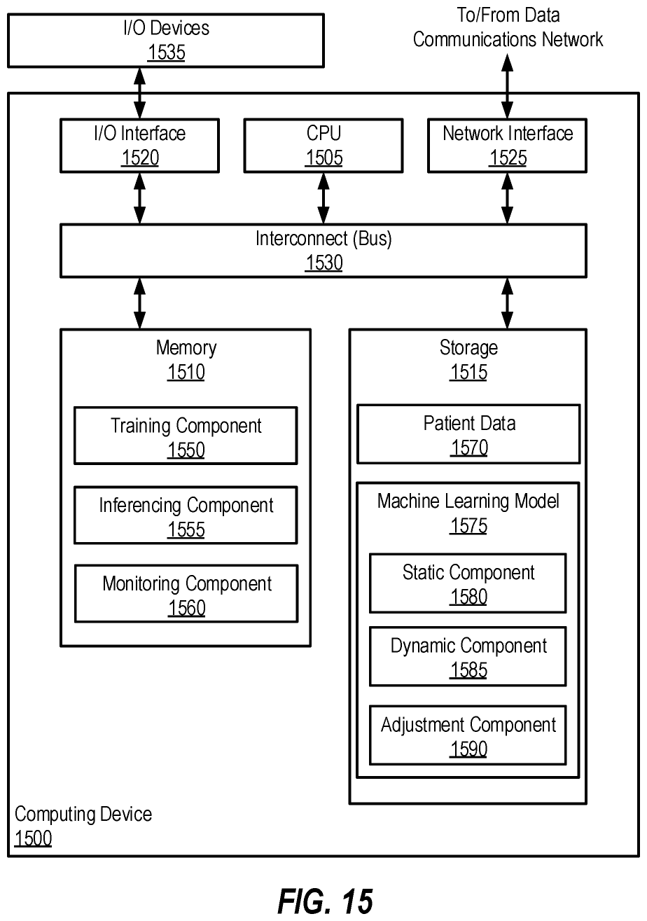

FIG. 15 depicts an example computing device 1500 configured to perform various aspects of the present disclosure. Although depicted as a physical device, in embodiments, the computing device 1500 may be implemented using virtual device(s), and/or across a number of devices (e.g., in a cloud environment). In one embodiment, the computing device 1500 corresponds to the machine learning system 135 of FIG. 1, the machine learning system 235 of FIG. 2, and/or the machine learning system 335 of FIG. 3.

As illustrated, the computing device 1500 includes a CPU 1505, memory 1510, storage 1515, a network interface 1525, and one or more I/O interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes a training component 1550, an inferencing component 1555, and a monitoring component 1560, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

In one embodiment, the training component 1550 is used to generate training data and/or to train machine learning models (e.g., based on historical training data), as discussed above. The inferencing component 1555 may generally be used to generate efficacy scores and/or generate or select medication plans for patients, as discussed above. The monitoring component 1560 may be configured to monitor patient conditions to enable online refinement of the models, as discussed above.

In the illustrated example, the storage 1515 includes patient data 1570 (which may correspond to historical data, such as historical data 105 of FIG. 1, patient data 205 of FIG. 2, patient data 305 of FIG. 3, and the like). The storage 1515 also includes one or more machine learning models 1575 (which may correspond to the machine learning model 140 of FIG. 1, and/or the machine learning model 410 of FIG. 4). In the illustrated example, the machine learning model is a hybrid model including one or more static components 1580 (e.g., the static component 415 of FIG. 4), one or more dynamic components 1585 (e.g., the dynamic component 420 of FIG. 4), and an adjustment component 1590 (e.g., the adjustment component 425 of FIG. 4). Although depicted as residing in storage 1515, the historical data 1570 and machine learning model 1575 may be stored in any suitable location, including memory 1510.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system 135, the machine learning system 235, and/or the machine learning system 335) or related data available in the cloud. For example, the machine learning system could execute on a computing system in the cloud and train and/or use machine learning models. In such a case, the machine learning system could train models to generate efficacy scores, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:

Clause 1: A method, comprising: receiving patient data describing a patient; identifying a medication to be evaluated with respect to the patient; generating an efficacy score by processing at least a subset of the patient data using a hybrid machine learning model comprising a static portion and a dynamic portion, wherein the efficacy score indicates predicted efficacy of the medication for the patient; and providing the medication for the patient based at least in part on the efficacy score.

Clause 2: The method of Clause 1, wherein: the static portion of the hybrid machine learning model is used to process a first subset of the patient data; and the dynamic portion of the hybrid machine learning model is used to process a second subset of the patient data.

Clause 3: The method of any one of Clauses 1-2, wherein generating the efficacy score comprises processing output of the static portion of the hybrid machine learning model using the dynamic portion of the hybrid machine learning model.

Clause 4: The method of any one of Clauses 1-3, wherein the hybrid machine learning model is a medication-specific model trained for the medication.

Clause 5: The method of any one of Clauses 1-4, wherein the hybrid machine learning model is a medication-agnostic model that generates the efficacy score based in part on processing an indication of the medication.

Clause 6: The method of any one of Clauses 1-5, further comprising: generating an adjustment factor based on previous medication history for the patient; and adjusting the efficacy score based on the adjustment factor.

Clause 7: The method of any one of Clauses 1-6, wherein the patient data comprises: at least one attribute corresponding to an age of the patient, at least one attribute corresponding to a sex of the patient, at least one attribute corresponding to allergies of the patient, at least one attribute corresponding to diagnoses of the patient, at least one attribute corresponding to medications used by the patient, and at least one attribute corresponding to assistance needed by the patient.

Clause 8: The method of any one of Clauses 1-7, wherein the patient data comprises natural language text describing the patient, the method further comprising: generating a vector representation of the natural language text.

Clause 9: The method of any one of Clauses 1-8, the method further comprising, prior to generating the vector representation: normalizing the natural language text; and removing noise from the normalized natural language text.

Clause 10: A method, comprising: removing noise from the normalized natural language text. identifying a medication consumed by the patient; determining an efficacy of the medication based at least in part on whether the medication was successful in treating a disorder of the patient; training a hybrid machine learning model comprising a static portion and a dynamic portion based on at least a subset of the patient data and the determined efficacy of the medication; and deploying the hybrid machine learning model.

Clause 11: The method of Clause 10, wherein: the static portion of the hybrid machine learning model is used to process a first subset of the patient data; and the dynamic portion of the hybrid machine learning model is used to process a second subset of the patient data.

Clause 12: The method of any one of Clauses 10-11, wherein the hybrid machine learning model processes output of the static portion of the hybrid machine learning model using the dynamic portion of the hybrid machine learning model.

Clause 13: The method of any one of Clauses 10-12, wherein determining the efficacy of the medication comprises: determining that the medication successfully treated the disorder of the patient; and determining a length of time that elapsed before the medication successfully treated the disorder of the patient.

Clause 14: The method of any one of Clauses 10-13, wherein the hybrid machine learning model is a medication-specific model trained for the medication.

Clause 15: The method of any one of Clauses 10-14, wherein the hybrid machine learning model is a medication-agnostic model that receives an indication of the medication as input.

Clause 16: The method of any one of Clauses 10-15, further comprising: generating an adjustment portion of the hybrid machine learning model, wherein the adjustment portion generates adjustment factors based on previous medication history for the patient.

Clause 17: The method of any one of Clauses 10-16, wherein the patient data comprises: at least one attribute corresponding to an age of the patient, at least one attribute corresponding to a sex of the patient, at least one attribute corresponding to allergies of the patient, at least one attribute corresponding to diagnoses of the patient, at least one attribute corresponding to medications used by the patient, and at least one attribute corresponding to assistance needed by the patient.

Clause 18: The method of any one of Clauses 10-17, wherein the patient data comprises natural language text describing the patient, the method further comprising: generating a vector representation of the natural language text.

Clause 19: The method of any one of Clauses 10-18, the method further comprising, prior to generating the vector representation: normalizing the natural language text; and removing noise from the normalized natural language text.

Clause 20: A system, comprising: a memory comprising computer-executable instructions; and one or more processors configured to execute the computer-executable instructions and cause the processing system to perform a method in accordance with any one of Clauses 1-19.

Clause 21: A system, comprising means for performing a method in accordance with any one of Clauses 1-19.

Clause 22: A non-transitory computer-readable medium comprising computer-executable instructions that, when executed by one or more processors of a processing system, cause the processing system to perform a method in accordance with any one of Clauses 1-19.

Clause 23: A computer program product embodied on a computer-readable storage medium comprising code for performing a method in accordance with any one of Clauses 1-19.

What is claimed is:

1. A method, comprising:
receiving patient data describing a patient;
identifying a medication to be evaluated with respect to the patient;
generating an efficacy score by processing at least a subset of the patient data using a hybrid machine learning model comprising a static portion and a dynamic portion, wherein the efficacy score indicates predicted efficacy of the medication for the patient; and
providing the medication for the patient based at least in part on the efficacy score.

2. The method of claim 1, wherein:
the static portion of the hybrid machine learning model is used to process a first subset of the patient data; and
the dynamic portion of the hybrid machine learning model is used to process a second subset of the patient data.

3. The method of claim 2, wherein generating the efficacy score comprises processing output of the static portion of the hybrid machine learning model using the dynamic portion of the hybrid machine learning model.

4. The method of claim 1, wherein the hybrid machine learning model is a medication-specific model trained for the medication.

5. The method of claim 1, wherein the hybrid machine learning model is a medication-agnostic model that generates the efficacy score based in part on processing an indication of the medication.

6. The method of claim 1, further comprising:
generating an adjustment factor based on previous medication history for the patient; and
adjusting the efficacy score based on the adjustment factor.

7. The method of claim 1, wherein the patient data comprises:
at least one attribute corresponding to an age of the patient,
at least one attribute corresponding to a sex of the patient,
at least one attribute corresponding to allergies of the patient,
at least one attribute corresponding to diagnoses of the patient,
at least one attribute corresponding to medications used by the patient, and
at least one attribute corresponding to assistance needed by the patient.

8. The method of claim 1, wherein the patient data comprises natural language text describing the patient, the method further comprising:
generating a vector representation of the natural language text.

9. The method of claim 8, the method further comprising, prior to generating the vector representation:
normalizing the natural language text; and
removing noise from the normalized natural language text.

10. A method, comprising:
receiving patient data describing a patient;
identifying a medication consumed by the patient;
determining an efficacy of the medication based at least in part on whether the medication was successful in treating a disorder of the patient;
training a hybrid machine learning model comprising a static portion and a dynamic portion based on at least a subset of the patient data and the determined efficacy of the medication; and
deploying the hybrid machine learning model.

11. The method of claim 10, wherein:
the static portion of the hybrid machine learning model is used to process a first subset of the patient data; and
the dynamic portion of the hybrid machine learning model is used to process a second subset of the patient data.

12. The method of claim 11, wherein the hybrid machine learning model processes output of the static portion of the hybrid machine learning model using the dynamic portion of the hybrid machine learning model.

13. The method of claim 10, wherein determining the efficacy of the medication comprises:
determining that the medication successfully treated the disorder of the patient; and
determining a length of time that elapsed before the medication successfully treated the disorder of the patient.

14. The method of claim 10, wherein the hybrid machine learning model is a medication-specific model trained for the medication.

15. The method of claim 10, wherein the hybrid machine learning model is a medication-agnostic model that receives an indication of the medication as input.

16. The method of claim 10, further comprising:
generating an adjustment portion of the hybrid machine learning model, wherein the adjustment portion generates adjustment factors based on previous medication history for the patient.

17. The method of claim 10, wherein the patient data comprises:
at least one attribute corresponding to an age of the patient,
at least one attribute corresponding to a sex of the patient,
at least one attribute corresponding to allergies of the patient,
at least one attribute corresponding to diagnoses of the patient,
at least one attribute corresponding to medications used by the patient, and
at least one attribute corresponding to assistance needed by the patient.

18. The method of claim 10, wherein the patient data comprises natural language text describing the patient, the method further comprising:
generating a vector representation of the natural language text.

19. The method of claim 18, the method further comprising, prior to generating the vector representation:
normalizing the natural language text; and
removing noise from the normalized natural language text.

20. A non-transitory computer-readable storage medium comprising computer-readable program code that, when executed using one or more computer processors, performs an operation comprising:
receiving patient data describing a patient;
identifying a medication to be evaluated with respect to the patient;
generating an efficacy score by processing at least a subset of the patient data using a hybrid machine learning model comprising a static portion and a dynamic portion, wherein the efficacy score indicates predicted efficacy of the medication for the patient; and
providing the medication for the patient based at least in part on the efficacy score.

21. A method, comprising:
receiving patient data describing a patient;
identifying a medication to be evaluated with respect to the patient;
generating an efficacy score by processing at least a subset of the patient data using an efficacy model, wherein the efficacy score indicates predicted efficacy of the medication for the patient; and
providing the medication for the patient based at least in part on the efficacy score.
